SEQUENCE LISTING (120 sequences)

SEQ ID NO: 1 - protein, 8 aa, synthetic construct
  REGION 1..8: note = Synthetic: CDR-H1 (Chothia), Anti-HLA-G
GYSILSGY

SEQ ID NO: 2 - protein, 7 aa, synthetic construct
  REGION 1..7: note = Synthetic: CDR-H1 (Chothia), Nivolumab (PD-1)
GITFSNS

SEQ ID NO: 3 - protein, 7 aa, synthetic construct
  REGION 1..7: note = Synthetic: CDR-H1 (Chothia), Pembrolizumab (PD-1)
GYTFTNY

SEQ ID NO: 4 - protein, 7 aa, synthetic construct
  REGION 1..7: note = Synthetic: CDR-H1 (Chothia), Cemiplimab (PD-1)
GFTFSNF

SEQ ID NO: 5 - protein, 7 aa, synthetic construct
  REGION 1..7: note = Synthetic: CDR-H1 (Chothia), Dostarlimab (PD-1)
GFTFSSY

SEQ ID NO: 6 - protein, 7 aa, synthetic construct
  REGION 1..7: note = Synthetic: CDR-H1 (Chothia), Atezolizumab (PD-L1)
GFTFSDS

SEQ ID NO: 7 - protein, 7 aa, synthetic construct
  REGION 1..7: note = Synthetic: CDR-H1 (Chothia), Avelumab (PD-L1)
GFTFSSY

SEQ ID NO: 8 - protein, 7 aa, synthetic construct
  REGION 1..7: note = Synthetic: CDR-H1 (Chothia), Durvalumab (PD-L1)
GFTFSRY

SEQ ID NO: 9 - protein, 7 aa, synthetic construct
  REGION 1..7: note = Synthetic: CDR-H1 (Chothia), Cetuximab (EGFR)
GFSLTNY

SEQ ID NO: 10 - protein, 7 aa, synthetic construct
  REGION 1..7: note = Synthetic: CDR-H1 (Chothia), Magrolimab (CD47)
GYTFTNY

SEQ ID NO: 11 - protein, 6 aa, synthetic construct
  REGION 1..6: note = Synthetic: CDR-H1 (Kabat), Anti-HLA-G
SGYYWF

SEQ ID NO: 12 - protein, 5 aa, synthetic construct
  REGION 1..5: note = Synthetic: CDR-H1 (Kabat), Nivolumab (PD-1)
NSGMH

SEQ ID NO: 13 - protein, 5 aa, synthetic construct
  REGION 1..5: note = Synthetic: CDR-H1 (Kabat), Pembrolizumab (PD-1)
NYYMY

SEQ ID NO: 14 - protein, 5 aa, synthetic construct
  REGION 1..5: note = Synthetic: CDR-H1 (Kabat), Cemiplimab (PD-1)
NFGMT

SEQ ID NO: 15 - protein, 5 aa, synthetic construct
  REGION 1..5: note = Synthetic: CDR-H1 (Kabat), Dostarlimab (PD-1)
SYDMS

SEQ ID NO: 16 - protein, 5 aa, synthetic construct
  REGION 1..5: note = Synthetic: CDR-H1 (Kabat), Atezolizumab (PD-L1)
DSWIH

SEQ ID NO: 17 - protein, 5 aa, synthetic construct
  REGION 1..5: note = Synthetic: CDR-H1 (Kabat), Avelumab (PD-L1)
SYIMM

SEQ ID NO: 18 - protein, 5 aa, synthetic construct
  REGION 1..5: note = Synthetic: CDR-H1 (Kabat), Durvalumab (PD-L1)
RYWMS

SEQ ID NO: 19 - protein, 5 aa, synthetic construct
  REGION 1..5: note = Synthetic: CDR-H1 (Kabat), Cetuximab (EGFR)
NYGVH

SEQ ID NO: 20 - protein, 5 aa, synthetic construct
  REGION 1..5: note = Synthetic: CDR-H1 (Kabat), Magrolimab (CD47)
NYNMH

SEQ ID NO: 21 - protein, 5 aa, synthetic construct
  REGION 1..5: note = Synthetic: CDR-H2 (Chothia), Anti-HLA-G
YHSGS

SEQ ID NO: 22 - protein, 6 aa, synthetic construct
  REGION 1..6: note = Synthetic: CDR-H2 (Chothia), Nivolumab (PD-1)
WYDGSK

SEQ ID NO: 23 - protein, 6 aa, synthetic construct
  REGION 1..6: note = Synthetic: CDR-H2 (Chothia), Pembrolizumab (PD-1)
NPSNGG

SEQ ID NO: 24 - protein, 6 aa, synthetic construct
  REGION 1..6: note = Synthetic: CDR-H2 (Chothia), Cemiplimab (PD-1)
SGGGRD

SEQ ID NO: 25 - protein, 6 aa, synthetic construct
  REGION 1..6: note = Synthetic: CDR-H2 (Chothia), Dostarlimab (PD-1)
SGGGSY

SEQ ID NO: 26 - protein, 6 aa, synthetic construct
  REGION 1..6: note = Synthetic: CDR-H2 (Chothia), Atezolizumab (PD-L1)
SPYGGS

SEQ ID NO: 27 - protein, 6 aa, synthetic construct
  REGION 1..6: note = Synthetic: CDR-H2 (Chothia), Avelumab (PD-L1)
YPSGGI

SEQ ID NO: 28 - protein, 6 aa, synthetic construct
  REGION 1..6: note = Synthetic: CDR-H2 (Chothia), Durvalumab (PD-L1)
KQDGSE

SEQ ID NO: 29 - protein, 5 aa, synthetic construct
  REGION 1..5: note = Synthetic: CDR-H2 (Chothia), Cetuximab (EGFR)
WSGGN

SEQ ID NO: 30 - protein, 6 aa, synthetic construct
  REGION 1..6: note = Synthetic: CDR-H2 (Chothia), Magrolimab (CD47)
YPGNDD

SEQ ID NO: 31 - protein, 16 aa, synthetic construct
  REGION 1..16: note = Synthetic: CDR-H2 (Kabat), Anti-HLA-G
GIYHSGSTAYNPSLKS

SEQ ID NO: 32 - protein, 17 aa, synthetic construct
  REGION 1..17: note = Synthetic: CDR-H2 (Kabat), Nivolumab (PD-1)
VIWYDGSKRYYADSVKG

SEQ ID NO: 33 - protein, 17 aa, synthetic construct
  REGION 1..17: note = Synthetic: CDR-H2 (Kabat), Pembrolizumab (PD-1)
GINPSNGGTNFNEKFKN

SEQ ID NO: 34 - protein, 17 aa, synthetic construct
  REGION 1..17: note = Synthetic: CDR-H2 (Kabat), Cemiplimab (PD-1)
GISGGGRDTYFADSVKG

SEQ ID NO: 35 - protein, 17 aa, synthetic construct
  REGION 1..17: note = Synthetic: CDR-H2 (Kabat), Dostarlimab (PD-1)
TISGGGSYTYYQDSVKG

SEQ ID NO: 36 - protein, 17 aa, synthetic construct
  REGION 1..17: note = Synthetic: CDR-H2 (Kabat), Atezolizumab (PD-L1)
WISPYGGSTYYADSVKG

SEQ ID NO: 37 - protein, 17 aa, synthetic construct
  REGION 1..17: note = Synthetic: CDR-H2 (Kabat), Avelumab (PD-L1)
SIYPSGGITFYADTVKG

SEQ ID NO: 38 - protein, 17 aa, synthetic construct
  REGION 1..17: note = Synthetic: CDR-H2 (Kabat), Durvalumab (PD-L1)
NIKQDGSEKYYVDSVKG

SEQ ID NO: 39 - protein, 16 aa, synthetic construct
  REGION 1..16: note = Synthetic: CDR-H2 (Kabat), Cetuximab (EGFR)
VIWSGGNTDYNTPFTS

SEQ ID NO: 40 - protein, 17 aa, synthetic construct
  REGION 1..17: note = Synthetic: CDR-H2 (Kabat), Magrolimab (CD47)
TIYPGNDDTSYNQKFKD

SEQ ID NO: 41 - protein, 13 aa, synthetic construct
  REGION 1..13: note = Synthetic: CDR-H3, Anti-HLA-G
GGEVTYSRGPLDV

SEQ ID NO: 42 - protein, 4 aa, synthetic construct
  REGION 1..4: note = Synthetic: CDR-H3, Nivolumab (PD-1)
NDDY

SEQ ID NO: 43 - protein, 11 aa, synthetic construct
  REGION 1..11: note = Synthetic: CDR-H3, Pembrolizumab (PD-1)
RDYRFDMGFDY

SEQ ID NO: 44 - protein, 8 aa, synthetic construct
  REGION 1..8: note = Synthetic: CDR-H3, Cemiplimab (PD-1)
WGNIYFDY

SEQ ID NO: 45 - protein, 7 aa, synthetic construct
  REGION 1..7: note = Synthetic: CDR-H3, Dostarlimab (PD-1)
PYYAMDY

SEQ ID NO: 46 - protein, 9 aa, synthetic construct
  REGION 1..9: note = Synthetic: CDR-H3, Atezolizumab (PD-L1)
RHWPGGFDY

SEQ ID NO: 47 - protein, 11 aa, synthetic construct
  REGION 1..11: note = Synthetic: CDR-H3, Avelumab (PD-L1)
IKLGTVTTVDY

SEQ ID NO: 48 - protein, 12 aa, synthetic construct
  REGION 1..12: note = Synthetic: CDR-H3, Durvalumab (PD-L1)
EGGWFGELAFDY

SEQ ID NO: 49 - protein, 11 aa, synthetic construct
  REGION 1..11: note = Synthetic: CDR-H3, Cetuximab (EGFR)
ALTYYDYEFAY

SEQ ID NO: 50 - protein, 8 aa, synthetic construct
  REGION 1..8: note = Synthetic: CDR-H3, Magrolimab (CD47)
GGYRAMDY

SEQ ID NO: 51 - protein, 12 aa, synthetic construct
  REGION 1..12: note = Synthetic: CDR-L1, Anti-HLA-G
RASNAVSSSYLA

SEQ ID NO: 52 - protein, 11 aa, synthetic construct
  REGION 1..11: note = Synthetic: CDR-L1, Nivolumab (PD-1)
RASQSVSSYLA

SEQ ID NO: 53 - protein, 15 aa, synthetic construct
  REGION 1..15: note = Synthetic: CDR-L1, Pembrolizumab (PD-1)
RASKGVSTSGYSYLH

SEQ ID NO: 54 - protein, 11 aa, synthetic construct
  REGION 1..11: note = Synthetic: CDR-L1, Cemiplimab (PD-1)
RASLSINTFLN

SEQ ID NO: 55 - protein, 11 aa, synthetic construct
  REGION 1..11: note = Synthetic: CDR-L1, Dostarlimab (PD-1)
KASQDVGTAVA

SEQ ID NO: 56 - protein, 11 aa, synthetic construct
  REGION 1..11: note = Synthetic: CDR-L1, Atezolizumab (PD-L1)
RASQDVSTAVA

SEQ ID NO: 57 - protein, 14 aa, synthetic construct
  REGION 1..14: note = Synthetic: CDR-L1, Avelumab (PD-L1)
TGTSSDVGGYNYVS

SEQ ID NO: 58 - protein, 12 aa, synthetic construct
  REGION 1..12: note = Synthetic: CDR-L1, Durvalumab (PD-L1)
RASQRVSSSYLA

SEQ ID NO: 59 - protein, 11 aa, synthetic construct
  REGION 1..11: note = Synthetic: CDR-L1, Cetuximab (EGFR)
RASQSIGTNIH

SEQ ID NO: 60 - protein, 16 aa, synthetic construct
  REGION 1..16: note = Synthetic: CDR-L1, Magrolimab (CD47)
RSSQSIVYSNGNTYLG

SEQ ID NO: 61 - protein, 7 aa, synthetic construct
  REGION 1..7: note = Synthetic: CDR-L2, Anti-HLA-G
GASYRAT

SEQ ID NO: 62 - protein, 7 aa, synthetic construct
  REGION 1..7: note = Synthetic: CDR-L2, Nivolumab (PD-1)
DASNRAT

SEQ ID NO: 63 - protein, 7 aa, synthetic construct
  REGION 1..7: note = Synthetic: CDR-L2, Pembrolizumab (PD-1)
LASYLES

SEQ ID NO: 64 - protein, 7 aa, synthetic construct
  REGION 1..7: note = Synthetic: CDR-L2, Cemiplimab (PD-1)
AASSLHG

SEQ ID NO: 65 - protein, 7 aa, synthetic construct
  REGION 1..7: note = Synthetic: CDR-L2, Dostarlimab (PD-1)
WASTLHT

SEQ ID NO: 66 - protein, 7 aa, synthetic construct
  REGION 1..7: note = Synthetic: CDR-L2, Atezolizumab (PD-L1)
SASFLYS

SEQ ID NO: 67 - protein, 7 aa, synthetic construct
  REGION 1..7: note = Synthetic: CDR-L2, Avelumab (PD-L1)
DVSNRPS

SEQ ID NO: 68 - protein, 7 aa, synthetic construct
  REGION 1..7: note = Synthetic: CDR-L2, Durvalumab (PD-L1)
DASSRAT

SEQ ID NO: 69 - protein, 7 aa, synthetic construct
  REGION 1..7: note = Synthetic: CDR-L2, Cetuximab (EGFR)
YASESIS

SEQ ID NO: 70 - protein, 7 aa, synthetic construct
  REGION 1..7: note = Synthetic: CDR-L2, Magrolimab (CD47)
KVSNRFS

SEQ ID NO: 71 - protein, 9 aa, synthetic construct
  REGION 1..9: note = Synthetic: CDR-L3, Anti-HLA-G
QQHSLYPPT

SEQ ID NO: 72 - protein, 9 aa, synthetic construct
  REGION 1..9: note = Synthetic: CDR-L3, Nivolumab (PD-1)
QQSSNWPRT

SEQ ID NO: 73 - protein, 9 aa, synthetic construct
  REGION 1..9: note = Synthetic: CDR-L3, Pembrolizumab (PD-1)
QHSRDLPLT

SEQ ID NO: 74 - protein, 9 aa, synthetic construct
  REGION 1..9: note = Synthetic: CDR-L3, Cemiplimab (PD-1)
QQSSNTPFT

SEQ ID NO: 75 - protein, 9 aa, synthetic construct
  REGION 1..9: note = Synthetic: CDR-L3, Dostarlimab (PD-1)
QHYSSYPWT

SEQ ID NO: 76 - protein, 9 aa, synthetic construct
  REGION 1..9: note = Synthetic: CDR-L3, Atezolizumab (PD-L1)
QQYLYHPAT

SEQ ID NO: 77 - protein, 10 aa, synthetic construct
  REGION 1..10: note = Synthetic: CDR-L3, Avelumab (PD-L1)
SSYTSSSTRV

SEQ ID NO: 78 - protein, 9 aa, synthetic construct
  REGION 1..9: note = Synthetic: CDR-L3, Durvalumab (PD-L1)
QQYGSLPWT

SEQ ID NO: 79 - protein, 9 aa, synthetic construct
  REGION 1..9: note = Synthetic: CDR-L3, Cetuximab (EGFR)
QQNNNWPTT

SEQ ID NO: 80 - protein, 9 aa, synthetic construct
  REGION 1..9: note = Synthetic: CDR-L3, Magrolimab (CD47)
FQGSHVPYT

SEQ ID NO: 81 - protein, 122 aa, synthetic construct
  REGION 1..122: note = Synthetic: VH, Anti-HLA-G
QVQLQESGPGLVKPSETLSLTCAVSGYSILSGYYWFWIRQPPGKGLEWIGGIYHSGSTAYNPSLKSRVTISVDTSKNQFSLKLSSVTAADTAVYYCARGGEVTYSRGPLDVWGQGTTVTVSS

SEQ ID NO: 82 - protein, 113 aa, synthetic construct [sequence omitted]

SEQ ID NO: 83 - protein, 120 aa, synthetic construct [sequence omitted]

SEQ ID NO: 84 - protein, 117 aa, synthetic construct [sequence omitted]

SEQ ID NO: 85 - protein, 116 aa, synthetic construct
  REGION 1..116: note = Synthetic: VH, Dostarlimab (PD-1)
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSS

SEQ ID NO: 86 - protein, 118 aa, synthetic construct [sequence omitted]

SEQ ID NO: 87 - protein, 120 aa, synthetic construct
  REGION 1..120: note = Synthetic: VH, Avelumab (PD-L1)
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 88 - protein, 121 aa, synthetic construct
  REGION 1..121: note = Synthetic: VH, Durvalumab (PD-L1)
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 89 - protein, 119 aa, synthetic construct
  REGION 1..119: note = Synthetic: VH, Cetuximab (EGFR)
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 90 - protein, 117 aa, synthetic construct
  REGION 1..117: note = Synthetic: VH, Magrolimab (CD47)
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 91 - protein, 108 aa, synthetic construct
  REGION 1..108: note = Synthetic: VL, Anti-HLA-G
EIVLTQSPGTLSLSPGERATLSCRASNAVSSSYLAWYQQKPGQAPRLLIYGASYRATGIPDRFSGSGSGTDFTLTISRLEPEDFAVYYCQQHSLYPPTFGGGTKVEIK

SEQ ID NO: 92 - protein, 107 aa, synthetic construct [sequence omitted]

SEQ ID NO: 93 - protein, 111 aa, synthetic construct [sequence omitted]

SEQ ID NO: 94 - protein, 107 aa, synthetic construct [sequence omitted]

SEQ ID NO: 95 - protein, 107 aa, synthetic construct
  REGION 1..107: note = Synthetic: VL, Dostarlimab (PD-1)
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIK

SEQ ID NO: 96 - protein, 107 aa, synthetic construct [sequence omitted]

SEQ ID NO: 97 - protein, 110 aa, synthetic construct
  REGION 1..110: note = Synthetic: VL, Avelumab (PD-L1)
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 98 - protein, 108 aa, synthetic construct
  REGION 1..108: note = Synthetic: VL, Durvalumab (PD-L1)
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 99 - protein, 107 aa, synthetic construct
  REGION 1..107: note = Synthetic: VL, Cetuximab (EGFR)
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 100 - protein, 112 aa, synthetic construct
  REGION 1..112: note = Synthetic: VL, Magrolimab (CD47)
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 101 - protein, 452 aa, synthetic construct
  REGION 1..452: note = Synthetic: HC, Anti-HLA-G
QVQLQESGPGLVKPSETLSLTCAVSGYSILSGYYWFWIRQPPGKGLEWIGGIYHSGSTAYNPSLKSRVTISVDTSKNQFSLKLSSVTAADTAVYYCARGGEVTYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 440 aa, synthetic construct
  REGION 1..440: note = Synthetic: HC, Nivolumab (PD-1)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 103 - protein, 447 aa, synthetic construct
  REGION 1..447: note = Synthetic: HC, Pembrolizumab (PD-1)
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 104 - protein, 444 aa, synthetic construct
  REGION 1..444: note = Synthetic: HC, Cemiplimab (PD-1)
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 105 - protein, 443 aa, synthetic construct
  REGION 1..443: note = Synthetic: HC, Dostarlimab (PD-1)
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 106 - protein, 448 aa, synthetic construct
  REGION 1..448: note = Synthetic: HC, Atezolizumab (PD-L1)
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 450 aa, synthetic construct
  REGION 1..450: note = Synthetic: HC, Avelumab (PD-L1)
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 451 aa, synthetic construct
  REGION 1..451: note = Synthetic: HC, Durvalumab (PD-L1)
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 449 aa, synthetic construct
  REGION 1..449: note = Synthetic: HC, Cetuximab (EGFR)
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 444 aa, synthetic construct
  REGION 1..444: note = Synthetic: HC, Magrolimab (CD47)
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 111 - protein, 215 aa, synthetic construct
  REGION 1..215: note = Synthetic: LC, Anti-HLA-G
EIVLTQSPGTLSLSPGERATLSCRASNAVSSSYLAWYQQKPGQAPRLLIYGASYRATGIPDRFSGSGSGTDFTLTISRLEPEDFAVYYCQQHSLYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 214 aa, synthetic construct
  REGION 1..214: note = Synthetic: LC, Nivolumab (PD-1)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 218 aa, synthetic construct
  REGION 1..218: note = Synthetic: LC, Pembrolizumab (PD-1)
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 214 aa, synthetic construct
  REGION 1..214: note = Synthetic: LC, Cemiplimab (PD-1)
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 214 aa, synthetic construct
  REGION 1..214: note = Synthetic: LC, Dostarlimab (PD-1)
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 214 aa, synthetic construct
  REGION 1..214: note = Synthetic: LC, Atezolizumab (PD-L1)
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 216 aa, synthetic construct
  REGION 1..216: note = Synthetic: LC, Avelumab (PD-L1)
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 118 - protein, 215 aa, synthetic construct
  REGION 1..215: note = Synthetic: LC, Durvalumab (PD-L1)
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 214 aa, synthetic construct
  REGION 1..214: note = Synthetic: LC, Cetuximab (EGFR)
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 219 aa, synthetic construct
  REGION 1..219: note = Synthetic: LC, Magrolimab (CD47)
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0223363 A1
(43) Pub. Date: Jul. 10, 2025

(54) COMBINATION THERAPY INVOLVING ANTI-HLA-G ANTIBODIES AND ANTI-EGFR ANTIBODIES, ANTI-PD1 OR ANTI-PD-L1 ANTIBODIES, AND/OR ANTI-CD47

(71) Applicant: TIZONA THERAPEUTICS, INC., South San Francisco, CA (US)

(72) Inventors: Courtney Beers, South San Francisco, CA (US); Doug Hodges, South San Francisco, CA (US); Shelby Joe, South San Francisco, CA (US)

(21) Appl. No.: 18/853,967

(22) PCT Filed: Apr. 7, 2023
(86) PCT No.: PCT/US23/17798

§ 371 Date: Oct. 3, 2024

Related U.S. Application Data
(60) Provisional application No. 63/329,065, filed on Apr. 8, 2022.

(51) Int. Cl. C07K 16/28

(52) U.S. Cl. CPC C07K 16/2833; C07K 16/2803; C07K 16/2818; C07K 16/2827; C07K 16/2863; C07K 16/2896; C07K 2317/24

(57) ABSTRACT

Provided herein are combination therapies involving antibodies with binding specificity for HLA-G and antibodies with binding specificity for EGFR, PD-1 and/or PD-L1, and/or CD47 or SIRPα.

### RELATED APPLICATIONS

This application claims priority to U.S. Provisional Application No. 63/329,065 filed Apr. 8, 2022, the contents of which are incorporated herein by reference in its entirety.

### FIELD

Provided herein are combination therapies involving antibodies with binding specificity for HLA-G and antibodies with binding specificity for EGFR, PD-1 and/or PD-L1, and/or CD47 or SIRPα.

### SEQUENCE LISTING

The instant application contains a Sequence Listing which has been submitted electronically in .XML format and is hereby incorporated by reference in its entirety. The .XML copy, created on Apr. 6, 2023, is named “1107368.00128.xml” and is 161,005 bytes in size. The sequence listing contained in this .XML file is part of the specification and is hereby incorporated by reference herein in its entirety.

### BACKGROUND

HLA-G histocompatibility antigen, class I, G, also known as human leukocyte antigen G (HLA-G), is a protein that in humans is encoded by the HLA-G gene. HLA-G belongs to the HLA nonclassical class I heavy chain paralogues. HLA-G is a heterodimer consisting of a heavy chain and a light chain (beta-2 microglobulin). There are membrane bound and soluble forms of HLA-G.

HLA-G is normally expressed at the maternal-fetal interface and other immune-privileged sites. HLA-G plays a role in immune tolerance in pregnancy, being expressed in the placenta by extravillous trophoblast cells, while the classical MHC class I genes (HLA-A and HLA-B) are not. As HLA-G was first identified in placenta samples, many studies have evaluated its role in pregnancy disorders, such as preeclampsia and recurrent pregnancy loss. See, Michita, Rafael Tomoya et al., Human Immunology. 2016, 77 (10): 892-897, which is incorporated by reference herein in its entirety, including any drawings.

HLA-G has been shown to be immune-suppressive. By binding receptors expressed on various myeloid and lymphoid cells, HLA-G may directly inhibit the functions of NK cells, cytotoxic T-lymphocytes, B cells, neutrophils, monocytes, macrophages, and dendritic cells. HLA-G also inhibits T and NK cell proliferation and cytolytic activities. HLA-G suppresses phagocytosis and induces the generation or expansion of regulatory T cells.

HLA-G mediates immune function through at least three ITIM-containing inhibitory receptors, ILT2, ILT4, and KIR2DL4. On lymphoid and myeloid cells, for example, HLA-G mediates function through ILT2. On myeloid cells, HLA-G mediates function through ILT4. On decidual NK cells, HLA-G mediates immune function through KIR2DL4 and ILT2.

HLA-G is an immune checkpoint target. HLA-G can directly inhibit immune cell function through receptor binding and/or trogocytosis and impairment of chemotaxis. HLA-G can lend tumor cells a higher invasive and metastatic potential. HLA-G promotes evasion of tumor immune surveillance, and enhances metastasis and the progression of malignancies. During tumor progression HLA-G has other effects, such as, inhibition of immune cell cytolysis, induction of immune cell apoptosis, and/or the generation of regulatory cells through receptor binding and/or trogocytosis.

HLA-G expression is upregulated on a broad spectrum of tumors and is associated with poor prognosis and disease progression. Serum HLA-G levels are elevated in breast, lung, colorectal cancer (CRC), gastric, esophageal, neuroblastoma, cervical, and hematological cancers. HLA-G has also been found to be correlated with clinical parameters in advanced disease, such as, tumor metastasis, poor prognosis, immune escape, and tumor invasiveness.

PD-1 is a membrane protein of 268 amino acids. PD-1 includes an extracellular IgV domain, a transmembrane region, and an intracellular tail. The tail contains two phosphorylation sites located in an immunoreceptor tyrosine-based inhibitory motif and an immunoreceptor tyrosine-based switch motif. It has been suggested that PD-1 negatively regulates T-cell receptor signals.

PD-1 is moderately expressed on naive T cells, B cells, and NK cells and up-regulated by T/B cell receptor signaling on lymphocytes, monocytes, and myeloid cells. PD-1 has a role in regulating the immune system's response by down-regulating the immune system and promoting self-tolerance by suppressing T cell inflammatory activity. This prevents autoimmune diseases, but it can also prevent the immune system from killing cancer cells.

PD-1 is recognized as an important player in immune regulation and the maintenance of peripheral tolerance. PD-1 can be viewed as an immune checkpoint and operates through multiple different mechanisms. For example, PD-1 promotes apoptosis of antigen-specific T-cells in lymph nodes. Further, PD-1 reduces apoptosis in regulatory T cells (anti-inflammatory, suppressive T cells).

PD-1 binds two ligands, PD-L1 and PD-L2. Both PD-1 ligands are members of the CD28-B7 family of co-signaling molecules that play important roles throughout all stages of T-cell function and other cell functions. The interaction of PD-1 and its ligands sends a signal into the T cell and essentially switches it off or inhibits it.

Cancer cells take advantage of this system by driving high levels of expression of PD-L1. This allows cancer cells to gain control of the PD-1 pathway and switch off T cells expressing PD-1 thus suppressing the anticancer immune response. PD-L1 is correlated with poor prognosis in ovarian, renal, colorectal, pancreatic, liver cancers, and melanoma. Similarly, PD-1 expression on tumor infiltrating lymphocytes shows dysfunctional T cells in breast cancer and melanoma and correlates with poor prognosis in renal cancer.

PD-1 therapies which ‘unblock’ an existing immune response or which unblock the initiation of an immune response are effective but sometimes only a subgroup of subjects responds. In addition, even in the responding population the response is not always complete or optimal.

The epidermal growth factor receptor (EGFR; ErbB-1; HER1 in humans) is a transmembrane protein that is a receptor for members of the epidermal growth factor family (EGF family) of extracellular protein ligands. The epidermal growth factor receptor is a member of the ErbB family of receptors, a subfamily of four closely related receptor tyrosine kinases: EGFR (ErbB-1), HER2/neu (ErbB-2), Her 3 (ErbB-3), and Her 4 (ErbB-4). In many cancer types, mutations affecting EGFR expression or activity could result in more progressive cancer. Deficient signaling of the EGFR and other receptor tyrosine kinases in humans is associated with diseases such as Alzheimer's, while over-expression is associated with the development of a wide variety of tumors. Interruption of EGFR signalling, either by blocking EGFR binding sites on the extracellular domain of the receptor or by inhibiting intracellular tyrosine kinase activity, can prevent the growth of EGFR-expressing tumors and improve the patient's condition.

EGFR is a transmembrane protein that is activated by binding of its specific ligands, including epidermal growth factor and transforming growth factor α (TGFα). ErbB2 has no known direct activating ligand and may be in an activated state constitutively or become active upon heterodimerization with other family members such as EGFR. Upon activation by its growth factor ligands, EGFR undergoes a transition from an inactive monomeric form to an active homodimer.

EGFR dimerization stimulates its intrinsic intracellular protein-tyrosine kinase activity. As a result, autophosphorylation of several tyrosine (Y) residues in the C-terminal domain of EGFR occurs. These include Y992, Y1045, Y1068, Y1148, and Y1173. Autophosphorylation elicits downstream activation and signaling by several other proteins that associate with the phosphorylated tyrosines through their own phosphotyrosine-binding SH2 domains. These downstream signaling proteins initiate several signal transduction cascades, principally the MAPK, Akt, and JNK pathways, leading to DNA synthesis and cell proliferation. Such proteins modulate phenotypes such as cell migration, adhesion, and proliferation. The kinase domain of EGFR can also cross-phosphorylate tyrosine residues of other receptors it is aggregated with, and can itself be activated in that manner.

Mutations that lead to EGFR overexpression (known as upregulation or amplification) have been associated with a number of cancers, including adenocarcinoma of the lung (40% of cases), anal cancers, glioblastoma (50%), and epithelian tumors of the head and neck (80-100%). These somatic mutations involving EGFR lead to its constant activation, which produces uncontrolled cell division. In glioblastoma a specific mutation of EGFR, called EGFRvIII, is often observed. Mutations, amplifications, or misregulations of EGFR or family members are implicated in about 30% of all epithelial cancer.

CD47 (Cluster of Differentiation 47) also known as integrin associated protein (IAP) is a transmembrane protein that in humans is encoded by the CD47 gene. CD47 belongs to the immunoglobulin superfamily and partners with membrane integrins and also binds the ligands thrombospondin-1 (TSP-1) and signal-regulatory protein alpha (SIRPα). CD47 is involved in a range of cellular processes, including apoptosis, proliferation, adhesion, and migration. Furthermore, CD47 plays a key role in immune and angiogenic responses. CD47 is ubiquitously expressed in human cells and has been found to be overexpressed in many different tumor cells.

CD47 is a 50 kDa membrane receptor that has extracellular N-terminal IgV domain, five transmembrane domains, and a short C-terminal intracellular tail. There are four alternatively spliced isoforms of CD47 that differ only in the length of their cytoplasmic tail.

Form 2 is the most widely expressed form that is found in all circulating and immune cells. The second most abundant isoform is form 4, which is predominantly expressed in the brain and in the peripheral nervous system. Only keratinocytes express significant amounts of form 1.

CD47 is a high affinity receptor for thrombospondin-1 (TSP-1), a secreted glycoprotein that plays a role in vascular development and angiogenesis, and in this later capacity the TSP1/CD47 interaction inhibits nitric oxide signaling at multiple levels in vascular cells. Binding of TSP-1 to CD47 influences several fundamental cellular functions including cell migration and adhesion, cell proliferation, or apoptosis and plays a role in the regulation of angiogenesis and inflammation.

CD47 interacts with signal-regulatory protein alpha (SIRPα), an inhibitory transmembrane receptor present on myeloid cells. The CD47/SIRPα interaction leads to bidirectional signaling, resulting in different cell-to-cell responses including inhibition of phagocytosis, stimulation of cell-cell fusion, and T-cell activation. CD47 interacts with several membrane integrins, most commonly integrin αvβ₃. These interactions result in CD47/integrin complexes that affect a range of cell functions including adhesion, spreading, and migration.

CD47 was first identified as a tumor antigen on human ovarian cancer in the 1980s. Since then, CD47 has been found to be expressed on multiple human tumor types including acute myeloid leukemia (AML), chronic myeloid leukemia, acute lymphoblastic leukemia (ALL), non-Hodgkin's lymphoma (NHL), multiple myeloma (MM), bladder cancer, and other solid tumors. CD47 is also highly expressed on pediatric and adult brain tumors.

High levels of CD47 allows cancer cells to avoid phagocytosis. This is due to engagement of the SIRP-α on macrophages by CD47. Engagement of SIRP-α leads to inhibition of phagocytosis.

Anti-CD47 antibody-mediated phagocytosis of cancer by macrophages can initiate an antitumor T-cell immune response. Anti-CD47 antibody treatment not only enables macrophage phagocytosis of cancer cells, but also fosters the activation of cancer-specific lymphocytes: cancer cells display mutant proteins to which the immune system can now react.

Combination therapies involving antibodies with binding specificity for HLA-G and antibodies with binding specificity for EGFR, PD-1 and/or PD-L1, and/or CD47 or SIRPα are needed.

### SUMMARY

Provided herein are methods and pharmaceuticals compositions for treatment of a subject suffering from cancer comprising an antibody which binds to HLA-G and an antibody which binds to EGFR, PD-1 or PD-L1, and/or CD47 or SIRPα.

A first aspect provides a pharmaceutical composition, comprising an antibody which binds to HLA-G and an antibody which binds to an epidermal growth factor receptor (EGFR). In some embodiments, the antibody that binds to HLA-G comprises or consists of a heavy chain variable region (VH) and a light chain variable region (VL), with VH and/or VL comprising:
a) a VHCDR1 having the sequence set forth in SEQ ID NO: 1 or SEQ ID NO: 11,
b) a VHCDR2 having the sequence set forth in SEQ ID NO: 21 or SEQ ID NO: 31,
c) a VHCDR3 having the sequence set forth in SEQ ID NO: 41,
d) a VLCDR1 having the sequence set forth in SEQ ID NO: 51,
e) a VLCDR2 having the sequence set forth in SEQ ID NO: 61, and
f) a VLCDR3 having the sequence set forth in SEQ ID NO: 71.
   In some embodiments, the antibody that binds to HLA-G comprises or consists of a heavy chain variable region (VH) and a light chain variable region (VL), the VH comprising, consisting of, or consisting essentially of a VH having the sequence set forth in SEQ ID NO: 81 and the VL comprising, consisting of, or consisting essentially of a VL having the sequence set forth in SEQ ID NO: 91. In some embodiments, the antibody that binds to HLA-G is an antibody comprising a heavy chain and a light chain, the heavy chain comprising or consisting of one or more molecules having a sequence comprising or consisting of SEQ ID NO: 101 and the light chain comprising or consisting of one or more molecules having a sequence comprising or consisting of SEQ ID NO: 111.

In some embodiments, the antibody which binds to an epidermal growth factor receptor (EGFR) comprises or consists of a heavy chain variable region (VH) and a light chain variable region (VL), with VH and/or VL comprising:
a) a VHCDR1 having the sequence set forth in SEQ ID NO: 9 or SEQ ID NO: 19,
b) a VHCDR2 having the sequence set forth in SEQ ID NO: 29 or SEQ ID NO: 39,
c) a VHCDR3 having the sequence set forth in SEQ ID NO: SEQ ID NO: 49,
d) a VLCDR1 having the sequence set forth in SEQ ID NO: SEQ ID NO: 59,
e) a VLCDR2 having the sequence set forth in SEQ ID NO: SEQ NO: 69, and
f) a VLCDR3 having the sequence set forth in SEQ ID NO: 79.
   In some embodiments, the antibody which binds to an epidermal growth factor receptor (EGFR) comprises or consists of a heavy chain variable region (VH) and a light chain variable region (VL), VH and/or VL comprising, consisting of, or consisting essentially of a VH having the sequence set forth in SEQ ID NO: 89 and the VL comprising, consisting of, or consisting essentially of a VL having the sequence set forth in SEQ ID NO: 99. In some embodiments, the antibody that binds to epidermal growth factor receptor (EGFR) is an antibody comprising a heavy chain and a light chain, the heavy chain comprising or consisting of one or more molecules having a sequence comprising or consisting of SEQ ID NO: 109 and the light chain comprising or consisting of one or more molecules having a sequence comprising or consisting of SEQ ID NO: 119.

A second aspect provide a pharmaceutical composition, comprising an antibody which binds to HLA-G and an antibody which binds to PD-1 and/or PD-L1. In some embodiments, the antibody that binds to HLA-G comprises or consists of a heavy chain variable region (VH) and a light chain variable region (VL), with VH and/or VL comprising:
a) a VHCDR1 having the sequence set forth in SEQ ID NO: 1 or SEQ ID NO: 11,
b) a VHCDR2 having the sequence set forth in SEQ ID NO: 21 or SEQ ID NO: 31,
c) a VHCDR3 having the sequence set forth in SEQ ID NO: 41,
d) a VLCDR1 having the sequence set forth in SEQ ID NO: 51,
e) a VLCDR2 having the sequence set forth in SEQ ID NO: 61, and
f) a VLCDR3 having the sequence set forth in SEQ ID NO: 71.
   In some embodiments, the antibody that binds to HLA-G comprises or consists of a heavy chain variable region (VH) and a light chain variable region (VL), the VH comprising, consisting of, or consisting essentially of a VH having the sequence set forth in SEQ ID NO: 81 and the VL comprising, consisting of, or consisting essentially of a VL having the sequence set forth in SEQ ID NO: 91. In some embodiments, the antibody that binds to HLA-G is an antibody comprising a heavy chain and a light chain, the heavy chain comprising or consisting of one or more molecules having a sequence comprising or consisting of SEQ ID NO: 101 and the light chain comprising or consisting of one or more molecules having a sequence comprising or consisting of SEQ ID NO: 111.

In some embodiments, the antibody which binds to PD-1 and/or PD-L1 comprises or consists of a heavy chain variable region (VH) and a light chain variable region (VL), VH and/or VL comprising:
a) a VHCDR1 having the sequence set forth in SEQ ID NOs: 2-8 or SEQ ID NOs: 12-18,
b) a VHCDR2 having the sequence set forth in SEQ ID NOs: 22-28 or SEQ NOs 32-38,
c) a VHCDR3 having the sequence set forth in SEQ ID NOs: 42-48,
d) a VLCDR1 having the sequence set forth in SEQ ID NOs: 52-58,
e) a VLCDR2 having the sequence set forth in SEQ ID NOs: 62-68, and
f) a VLCDR3 having the sequence set forth in SEQ ID NOs: 72-78.
   In some embodiments, the antibody which binds to PD-1 and/or PD-L1 comprises or consists of a heavy chain variable region (VH) and a light chain variable region (VL), VH and/or VL comprising, consisting of, or consisting essentially of a VH having a sequence set forth in an one of SEQ ID NOs: SEQ NOs: 82-88 and a VL comprising, consisting of, or consisting essentially of a VL having a sequence set forth in any one of SEQ ID NOs: 92-98. In some embodiments, the antibody which binds to PD-1 and/or PD-L1 is an antibody comprising a heavy chain and a light chain, the heavy chain comprising or consisting of one or more molecules having a sequence comprising or consisting of a sequence set forth in any one of SEQ ID NOs: 102-108 and the light chain comprising or consisting of one or more molecules having a sequence comprising or consisting of a sequence set forth in any one of SEQ ID NOs: 112-118.

A third aspect provide an antibody which binds to HLA-G and an antibody which binds to CD47 or SIRPα. In some embodiments, the antibody that binds to HLA-G comprises or consists of a heavy chain variable region (VH) and a light chain variable region (VL), with VH and/or VL comprising:
a) a VHCDR1 having the sequence set forth in SEQ ID NO: 1 or SEQ ID NO: 11,
b) a VHCDR2 having the sequence set forth in SEQ ID NO: 21 or SEQ ID NO: 31,
c) a VHCDR3 having the sequence set forth in SEQ ID NO: 41,
d) a VLCDR1 having the sequence set forth in SEQ ID NO: 51,
e) a VLCDR2 having the sequence set forth in SEQ ID NO: 61, and
f) a VLCDR3 having the sequence set forth in SEQ ID NO: 71.
   In some embodiments, the antibody that binds to HLA-G comprises or consists of a heavy chain variable region (VH) and a light chain variable region (VL), the VH comprising, consisting of, or consisting essentially of a VH having the sequence set forth in SEQ ID NO: 81 and the VL comprising, consisting of, or consisting essentially of a VL having the sequence set forth in SEQ ID NO: 91. In some embodiments, the antibody that binds to HLA-G is an antibody comprising a heavy chain and a light chain, the heavy chain comprising or consisting of one or more molecules having a sequence comprising or consisting of SEQ ID NO: 101 and the light chain comprising or consisting of one or more molecules having a sequence comprising or consisting of SEQ ID NO: 111.

In some embodiments, the antibody which binds to an CD47 comprises or consists of a heavy chain variable region (VH) and a light chain variable region (VL), VH and/or VL comprising:
a) a VHCDR1 having the sequence set forth in SEQ ID NO: 10 or SEQ ID NO: 20,
b) a VHCDR2 having the sequence set forth in SEQ ID NO: 30 or SEQ ID NO: 40,
c) a VHCDR3 having the sequence set forth in SEQ ID NO: 50,
d) a VLCDR1 having the sequence set forth in SEQ ID NO: 60,
e) a VLCDR2 having the sequence set forth in SEQ ID NO: 70, and
f) a VLCDR3 having the sequence set forth in SEQ ID NO: 80.

In some embodiments, the antibody which binds to CD47 comprises or consists of a heavy chain variable region (VH) and a light chain variable region (VL), VH and/or VL comprising, consisting of, or consisting essentially of a VH having the sequence set forth in SEQ ID NO: 90 and of a VL comprising, consisting of, or consisting essentially of a VL having the sequence set forth in SEQ ID NO: 100. In some embodiments, the antibody which binds to CD47 is an antibody comprising a heavy chain and a light chain, the heavy chain comprising or consisting of one or more molecules having a sequence comprising or consisting of SEQ ID NO: 110 and the light chain comprising or consisting of one or more molecules having a sequence comprising or consisting of SEQ ID NO: 120.

A fourth aspect provides a method for treatment of a subject suffering from cancer, comprising administering to the subject any of the pharmaceutical compositions set forth herein. In some embodiments, the cancer is a solid cancer. In some embodiments, the cancer is a hematological cancer. In some embodiments, the subject is a human subject. In some embodiments, the method further comprises one or more of the following:
a) administering chemotherapy;
b) administering radiation therapy; and/or
c) administering one or more additional therapeutic agents.

A fifth aspect provides a method of modulating immune system function in a subject in need thereof, comprising the step of contacting a population of immune cells of the subject with any of the pharmaceutical compositions provided herein. In some embodiments, the subject is a human subject.

In some embodiments, the pharmaceutical composition is administered in an amount sufficient to achieve 1, 2, 3, 4, 5, 6, 7, or 8 of the following in the subject:
a) inhibition of immune suppression;
b) reduction of levels of regulatory T cells;
c) increase in activity of myeloid cells, cytotoxic T lymphocytes, helper T cells, NK cells, T cells, B cells, neutrophils, monocytes, macrophages, and/or dendritic cells;
d) increase in phagocytic activity;
e) inhibition of metastasis;
f) inhibition of tumor growth;
g) induction of tumor regression; and/or
h) enhancement of ILT2⁺ CD8⁺ T cell degranulation.

In some embodiments, the increase in phagocytic activity comprises an increase in percent phagocytosis as compared to a pharmaceutical composition with no antibody or a pharmaceutical composition with an antibody which binds HLA-G. In some embodiments, the method further comprises one or more of the following:
a) administering chemotherapy;
b) administering radiation therapy; and/or
c) administering one or more additional therapeutic agents.

### BRIEF DESCRIPTION OF THE DRAWINGS

FIG. 1 demonstrates an antibody that binds HLA-G (Fab fragment) in combination with cetuximab significantly increased macrophage phagocytosis of HLA-G⁺ A549 target cells compared to cetuximab alone. No activity was observed from the antibody that binds HLA-G compared to an untreated control. Data are shown as mean±standard deviation (n=2 replicates). **p<0.01 using students t-test.

FIG. 2A and FIG. 2B show an antibody that binds HLA-G in combination with pembrolizumab or atezolizumab, respectively, enhanced ILT2⁺CD8⁺ T cell degranulation (CD107a) above either single agent alone. An isotype matched antibody was used as a negative control. The graphs show the mean±standard deviation (n=2 replicate wells) from 1 representative donor.

FIG. 3A and FIG. 3B demonstrate an antibody that binds HLA-G in combination with magrolimab increased macrophage phagocytosis of endogenous HLA-G expressing HT-1376 and JEG-3 cells, respectively, compared to either single agent alone. FIG. 3C and FIG. 3D show that a triple-agent combination of an antibody that binds HLA-G together with magrolimab and cetuximab has greater activity than single or double-agent treatments to induce phagocytosis of A549 and SK-OV-3 cells engineered to express HLA-G. Isotype antibodies were used as negative controls. Data are shown as mean±standard deviation (n=2 replicates).

### DETAILED DESCRIPTION

Provided herein are combination therapies involving antibodies with binding specificity for HLA-G and antibodies with binding specificity for EGFR, PD-1 and/or PD-L1, and/or CD47 or SIRPα.

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The terms “HLA-G,” “human leukocyte antigen G,” and “HLA-G histocompatibility antigen, class I, G” are used interchangeably herein. Unless specified otherwise, the terms include any variants, isoforms, and species homologs of human HLA-G that are naturally expressed by cells, or that are expressed by cells transfected with a HLA-G gene.

The terms “PD-1,” “programmed cell death protein 1,” and “Cluster of Differentiation 279” are used interchangeably herein. Unless specified otherwise, the terms include any variants, isoforms, and species homologs of human PD-1 that are naturally expressed by cells, or that are expressed by cells transfected with a PD-1 gene.

The terms “PD-L1,” “programmed death-ligand 1,” “Cluster of Differentiation 274,” “B7 homolog 1,” and “B7-H1” are used interchangeably herein. Unless specified otherwise, the terms include any variants, isoforms, and species homologs of human PD-L1 that are naturally expressed by cells, or that are expressed by cells transfected with a PD-L1 gene.

The terms “EGFR,” “epidermal growth factor receptor,” “ErbB-1,” and “HER1” are used interchangeably herein. Unless specified otherwise, the terms include any variants, isoforms, and species homologs of human EGFR that are naturally expressed by cells, or that are expressed by cells transfected with a EGFR gene.

The terms “CD47,” “Cluster of Differentiation 47,” and “integrin associated protein” are used interchangeably herein. Unless specified otherwise, the terms include any variants, isoforms, and species homologs of human CD47 that are naturally expressed by cells, or that are expressed by cells transfected with the CD47 gene.

The terms “signal regulatory protein α” or “SIRPα” are used interchangebly herein.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, CH1, CH2, and CH3. Each light chain typically comprises a light chain variable region (VL) and a light chain constant region. The light chain constant region typically comprises one domain, abbreviated CL.

The term “antibody” describes a type of immunoglobulin molecule and is used herein in its broadest sense. An antibody specifically includes intact antibodies (e.g., intact immunoglobulins) and antibody fragments. Antibodies comprise at least one antigen-binding domain. One example of an antigen-binding domain is an antigen binding domain formed by a VH-VL dimer.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and confer antigen specificity and binding affinity to the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

Unless otherwise specified, the numbering scheme used for identification of a particular CDR herein is the Kabat numbering scheme. Variant and equivalent antibodies with a Chothia numbering scheme are intended to be within the scope of the invention.

TABLE 1
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments. In some embodiments, an antibody that binds HLA-G, an antibody that binds PD-1 and/or an antibody that binds PD-L1, an antibody that binds EGFR, and/or an antibody that binds CD47 or SIRPα includes antibody fragments of each of the specificied antibodies.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by papain digestion of a full-length antibody.

“F(ab′)₂” fragments contain two Fab′ fragments joined, near the hinge region, by disulfide bonds. F(ab′)₂ fragments may be generated, for example, by pepsin digestion of an intact antibody. The F(ab′) fragments can be dissociated, for example, by treatment with B-mercaptoethanol.

“Single-chain Fv” or “sFv” or “scFv” antibody fragments comprise a VH domain and a VL domain in a single polypeptide chain. The VH and VL are generally linked by a peptide linker. See Pluckthun A. (1994). Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety. “scFv-Fc” fragments comprise an scFv attached to an Fc domain. For example, an Fc domain may be attached to the C-terminal of the scFv. The Fc domain may follow the V_{H} or V_{L} depending on the orientation of the variable domains in the scFv (i.e., V_{H}-V_{L} or V_{L}-V_{H}). Any suitable Fc domain known in the art or described herein may be used.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

With regard to the binding of an antibody to a target molecule, the terms “binding” or “binds to” a particular antigen (e.g., a polypeptide target) or an epitope on a particular antigen mean binding that is measurably different from a non-selective interaction. Binding can be measured, for example, by determining binding of a molecule compared to binding of a control molecule. Binding can also be determined by competition with a control molecule that is similar to the target, such as an excess of non-labeled target. In that case, binding is indicated if the binding of the labeled target to a probe is competitively inhibited by the excess non-labeled target.

“Treating” or “treatment” of any cancer refers, in certain embodiments, to ameliorating a cancer that exists in a subject. In another embodiment, “treating” or “treatment” includes ameliorating at least one physical parameter, which may be indiscernible by the subject. In yet another embodiment, “treating” or “treatment” includes modulating the cancer, either physically (e.g., stabilization of a discernible symptom) or physiologically (e.g., stabilization of a physical parameter) or both.

As used herein, the term “subject” means a mammal or a human. In some embodiments subjects include, but are not limited to, monkeys, dogs, cats, mice, rats, cows, horses, camels, avians, goats, and sheep.

### 2. Antibody Combinations

Provided herein are methods and antibody combinations for the treatment of cancer or other diseases. The antibody combinations combine an antibody that binds HLA-G and antibodies with binding specificity for EGFR, PD-1 and/or PD-L1, and/or CD47 or SIRPα.

### CDR-H1+CDR-H2+CDR-H3 Regions of the Antibodies

In some embodiments, the antibody that binds to HLA-G comprises a VH sequence comprising a CDR-H1 sequence comprising, consisting of, or consisting essentially of a sequence selected from SEQ ID NO: 1 or SEQ ID NO: 11, a CDR-H2 sequence comprising, consisting of, or consisting essentially of a sequence selected from SEQ ID NO: 21 or SEQ ID NO: 31, and a CDR-H3 sequence comprising, consisting of, or consisting essentially of a sequence selected from SEQ ID NO: 41. In some embodiments, the CDR-H1 sequence, CDR-H2 sequence, and the CDR-H3 sequence are all from a single illustrative V_{H} sequence provided in this disclosure. For example, in some embodiments, the CDR-H1, CDR-H2, and CDR-H3 are all from a single illustrative V_{H} sequence having SEQ ID NO: 81.

In some embodiments, the antibody that binds to EGFR comprises a V_{H} sequence comprising a CDR-H1 sequence comprising, consisting of, or consisting essentially of a sequence selected from SEQ ID NO: 9 or SEQ ID NO: 19, a CDR-H2 sequence comprising, consisting of, or consisting essentially of a sequence selected from SEQ ID NO: 29 or SEQ ID NO: 39, and a CDR-H3 sequence comprising, consisting of, or consisting essentially of a sequence selected from SEQ ID NO: 49. In some embodiments, the CDR-H1 sequence, CDR-H2 sequence, and the CDR-H3 sequence are all from a single illustrative V_{H} sequence provided in this disclosure. For example, in some embodiments, the CDR-H1, CDR-H2, and CDR-H3 are all from a single illustrative V_{H} sequence selected from SEQ ID NO: 89.

In some embodiments, the antibody that binds to PD-1 comprises a V_{H} sequence comprising a CDR-H1 sequence comprising, consisting of, or consisting essentially of a sequence selected from SEQ ID NOs: 2-5 or SEQ ID NOs: 12-15, a CDR-H2 sequence comprising, consisting of, or consisting essentially of a sequence selected from SEQ ID NOs: 22-25 or SEQ ID NOs: 32-35, and a CDR-H3 sequence comprising, consisting of, or consisting essentially of a sequence selected from SEQ ID NOs: 42-45. In some embodiments, the CDR-H1 sequence, CDR-H2 sequence, and the CDR-H3 sequence are all from a single illustrative V_{H} sequence provided in this disclosure. For example, in some embodiments, the CDR-H1, CDR-H2, and CDR-H3 are all from a single illustrative V_{H} sequence selected from SEQ ID NOs: 82-85.

In some embodiments, the antibody that binds to PD-1 comprises a V_{H} sequence comprising a CDR-H1 sequence comprising, consisting of, or consisting essentially of a sequence comprising SEQ ID NO: 2 or SEQ ID NO: 12, a CDR-H2 sequence comprising, consisting of, or consisting essentially of a sequence comprising SEQ ID NO: 22 or SEQ ID NO: 32, and a CDR-H3 sequence comprising, consisting of, or consisting essentially of a sequence comprising SEQ ID NO: 42. In some embodiments, the antibody that binds to PD-1 comprises a V_{H} sequence comprising a CDR-H1 sequence comprising, consisting of, or consisting essentially of a sequence comprising SEQ ID NO: 3 or SEQ ID NO: 13, a CDR-H2 sequence comprising, consisting of, or consisting essentially of a sequence comprising SEQ ID NO: 23 or SEQ ID NO: 33, and a CDR-H3 sequence comprising, consisting of, or consisting essentially of a sequence comprising SEQ ID NO: 43. In some embodiments, the antibody that binds to PD-1 comprises a V_{H} sequence comprising a CDR-H1 sequence comprising, consisting of, or consisting essentially of a sequence comprising SEQ ID NO: 4 or SEQ ID NO: 14, a CDR-H2 sequence comprising, consisting of, or consisting essentially of a sequence comprising SEQ ID NO: 24 or SEQ ID NO: 34, and a CDR-H3 sequence comprising, consisting of, or consisting essentially of a sequence comprising SEQ ID NO: 44. In some embodiments, the antibody that binds to PD-1 comprises a V_{H} sequence comprising a CDR-H1 sequence comprising, consisting of, or consisting essentially of a sequence comprising SEQ ID NO: 5 or SEQ ID NO: 15, a CDR-H2 sequence comprising, consisting of, or consisting essentially of a sequence comprising SEQ ID NO: 25 or SEQ ID NO: 35, and a CDR-H3 sequence comprising, consisting of, or consisting essentially of a sequence comprising SEQ ID NO: 45.

In some embodiments, the CDR-H1 sequence, CDR-H2 sequence, and the CDR-H3 sequence are all from a single illustrative V_{H} sequence provided in this disclosure. For example, in some embodiments, the CDR-H1, CDR-H2, and CDR-H3 are all from a single V_{H} sequence having SEQ ID NO: 82. In some embodiments, the CDR-H1, CDR-H2, and CDR-H3 are all from a single V_{H} sequence having SEQ ID NO: 83. In some embodiments, the CDR-H1, CDR-H2, and CDR-H3 are all from a single V_{H} sequence having SEQ ID NO: 84. In some embodiments, the CDR-H1, CDR-H2, and CDR-H3 are all from a single V_{H} sequence having SEQ ID NO: 85.

In some embodiments, the antibody that binds to PD-L1 comprises a V_{H} sequence comprising a CDR-H1 sequence comprising, consisting of, or consisting essentially of a sequence selected from SEQ ID NOs: 6-8 or SEQ ID NOs: 16-18, a CDR-H2 sequence comprising, consisting of, or consisting essentially of a sequence selected from SEQ ID NOs: 26-28 or SEQ ID NOs: 36-38, and a CDR-H3 sequence comprising, consisting of, or consisting essentially of a sequence selected from SEQ ID NOs: 46-48. In some embodiments, the CDR-H1 sequence, CDR-H2 sequence, and the CDR-H3 sequence are all from a single illustrative V_{H} sequence provided in this disclosure. For example, in some embodiments, the CDR-H1, CDR-H2, and CDR-H3 are all from a single illustrative V_{H} sequence selected from SEQ ID NOs: 86-88.

In some embodiments, the antibody that binds to PD-L1 comprises a V_{H} sequence comprising a CDR-H1 sequence comprising, consisting of, or consisting essentially of a sequence comprising SEQ ID NO: 6 or SEQ ID NO: 16, a CDR-H2 sequence comprising, consisting of, or consisting essentially of a sequence comprising SEQ ID NO: 26 or SEQ ID NO: 36, and a CDR-H3 sequence comprising, consisting of, or consisting essentially of a sequence comprising SEQ ID NO: 46. In some embodiments, the antibody that binds to PD-L1 comprises a V_{H} sequence comprising a CDR-H1 sequence comprising, consisting of, or consisting essentially of a sequence comprising SEQ ID NO: 7 or SEQ ID NO: 17, a CDR-H2 sequence comprising, consisting of, or consisting essentially of a sequence comprising SEQ ID NO: 27 or SEQ ID NO: 37, and a CDR-H3 sequence comprising, consisting of, or consisting essentially of a sequence comprising SEQ ID NO: 47. In some embodiments, the antibody that binds to PD-L1 comprises a V_{H} sequence comprising a CDR-H1 sequence comprising, consisting of, or consisting essentially of a sequence comprising SEQ ID NO: 8 or SEQ ID NO: 18, a CDR-H2 sequence comprising, consisting of, or consisting essentially of a sequence comprising SEQ ID NO: 28 or SEQ ID NO: 38, and a CDR-H3 sequence comprising, consisting of, or consisting essentially of a sequence comprising SEQ ID NO: 48.

In some embodiments, the CDR-H1 sequence, CDR-H2 sequence, and the CDR-H3 sequence are all from a single illustrative V_{H} sequence provided in this disclosure. In some embodiments, the CDR-H1, CDR-H2, and CDR-H3 are all from a single V_{H} sequence having SEQ ID NO: 86. In some embodiments, the CDR-H1, CDR-H2, and CDR-H3 are all from a single V_{H} sequence having SEQ ID NO: 87. In some embodiments, the CDR-H1, CDR-H2, and CDR-H3 are all from a single V_{H} sequence having SEQ ID NO: 88.

In some embodiments, the antibody that binds to CD47 comprises a V_{H} sequence comprising a CDR-H1 sequence comprising, consisting of, or consisting essentially of SEQ ID NO: 10 or SEQ ID NO: 20, a CDR-H2 sequence comprising, consisting of, or consisting essentially of SEQ ID NO: 30 or SEQ ID NO: 40, and a CDR-H3 sequence comprising, consisting of, or consisting essentially of sequence SEQ ID NO: 50. In some embodiments, the CDR-H1 sequence, CDR-H2 sequence, and the CDR-H3 sequence are all from a single illustrative V_{H} sequence provided in this disclosure. For example, in some embodiments, the CDR-H1, CDR-H2, and CDR-H3 are all from a single illustrative V_{H} sequence having SEQ ID NO: 90.

In some embodiments, the antibody that binds to HLA-G comprises a V_{H} sequence comprising a CDR-H1 sequence comprising SEQ ID NO: 1 or SEQ ID NO: 11, a CDR-H2 sequence comprising SEQ ID NO: 21 or SEQ ID NO: 31, and a CDR-H3 sequence comprising SEQ ID NO: 41 and the antibody that binds to EGFR comprises a V_{H} sequence comprises a V_{H} sequence comprising a CDR-H1 sequence comprising SEQ ID NO: 9 or SEQ ID NO: 19, a CDR-H2 sequence comprising SEQ ID NO: 29 or SEQ ID NO: 39, and a CDR-H3 sequence comprising SEQ ID NO: 49.

In some embodiments, the antibody that binds to HLA-G comprises a V_{H} sequence comprising a CDR-H1 sequence comprising SEQ ID NO: 1 or SEQ ID NO: 11, a CDR-H2 sequence comprising SEQ ID NO: 21 or SEQ ID NO: 31, and a CDR-H3 sequence comprising SEQ ID NO: 41 and the antibody that binds to PD-1 comprises a V_{H} sequence comprises a V_{H} sequence comprising a CDR-H1 sequence comprising SEQ ID NO: 2 or SEQ ID NO: 12, a CDR-H2 sequence comprising SEQ ID NO: 22 or SEQ ID NO: 32, and a CDR-H3 sequence comprising SEQ ID NO: 42. In some embodiments, the antibody that binds to HLA-G comprises a V_{H} sequence comprising a CDR-H1 sequence comprising SEQ ID NO: 1 or SEQ ID NO: 11, a CDR-H2 sequence comprising SEQ ID NO: 21 or SEQ ID NO: 31, and a CDR-H3 sequence comprising SEQ ID NO: 41 and the antibody that binds to PD-1 comprises a V_{H} sequence comprising a CDR-H1 sequence comprising SEQ ID NO: 3 or SEQ ID NO: 13, a CDR-H2 sequence comprising SEQ ID NO: 23 or SEQ ID NO: 33, and a CDR-H3 sequence comprising SEQ ID NO: 43. In some embodiments, the antibody that binds to HLA-G comprises a V_{H} sequence comprising a CDR-H1 sequence comprising SEQ ID NO: 1 or SEQ ID NO: 11, a CDR-H2 sequence comprising SEQ ID NO: 21 or SEQ ID NO: 31, and a CDR-H3 sequence comprising SEQ ID NO: 41 and the antibody that binds to PD-1 comprises a V_{H} sequence comprising a CDR-H1 sequence comprising SEQ ID NO: 4 or SEQ ID NO: 14, a CDR-H2 sequence comprising SEQ ID NO: 24 or SEQ ID NO: 34, and a CDR-H3 sequence comprising SEQ ID NO: 44. In some embodiments, the antibody that binds to HLA-G comprises a V_{H} sequence comprising a CDR-H1 sequence comprising SEQ ID NO: 1 or SEQ ID NO: 11, a CDR-H2 sequence comprising SEQ ID NO: 21 or SEQ ID NO: 31, and a CDR-H3 sequence comprising SEQ ID NO: 41 and the antibody that binds to PD-1 comprises a V_{H} sequence comprising a CDR-H1 sequence comprising SEQ ID NO: 5 or SEQ ID NO: 15, a CDR-H2 sequence comprising SEQ ID NO: 25 or SEQ ID NO: 35, and a CDR-H3 sequence comprising SEQ ID NO: 45.

In some embodiments, the antibody that binds to HLA-G comprises a V_{H} sequence comprising a CDR-H1 sequence comprising SEQ ID NO: 1 or SEQ ID NO: 11, a CDR-H2 sequence comprising SEQ ID NO: 21 or SEQ ID NO: 31, and a CDR-H3 sequence comprising SEQ ID NO: 41 and the antibody that binds to PD-L1 comprises a V_{H} sequence comprising a CDR-H1 sequence comprising SEQ ID NO: 6 or SEQ ID NO: 16, a CDR-H2 sequence comprising SEQ ID NO: 26 or SEQ ID NO: 36, and a CDR-H3 sequence comprising SEQ ID NO: 46. In some embodiments, the antibody that binds to HLA-G comprises a V_{H} sequence comprising a CDR-H1 sequence comprising SEQ ID NO: 1 or SEQ ID NO: 11, a CDR-H2 sequence comprising SEQ ID NO: 21 or SEQ ID NO: 31, and a CDR-H3 sequence comprising SEQ ID NO: 41 and the antibody that binds to PD-L1 comprises a V_{H} sequence comprising a CDR-H1 sequence comprising SEQ ID NO: 7 or SEQ ID NO: 17, a CDR-H2 sequence comprising SEQ ID NO: 27 or SEQ ID NO: 37, and a CDR-H3 sequence comprising SEQ ID NO: 47. In some embodiments, the antibody that binds to HLA-G comprises a V_{H} sequence comprising a CDR-H1 sequence comprising SEQ ID NO: 1 or SEQ ID NO: 11, a CDR-H2 sequence comprising SEQ ID NO: 21 or SEQ ID NO: 31, and a CDR-H3 sequence comprising SEQ ID NO: 41 and the antibody that binds to PD-L1 comprises a V_{H} sequence comprising a CDR-H1 sequence comprising SEQ ID NO: 8 or SEQ ID NO: 18, a CDR-H2 sequence comprising SEQ ID NO: 28 or SEQ ID NO: 38, and a CDR-H3 sequence comprising SEQ ID NO: 48.

In some embodiments, the antibody that binds to HLA-G comprises a V_{H} sequence comprising a CDR-H1 sequence comprising SEQ ID NO: 1 or SEQ ID NO: 11, a CDR-H2 sequence comprising SEQ ID NO: 21 or SEQ ID NO: 31, and a CDR-H3 sequence comprising SEQ ID NO: 41 and the antibody that binds to CD47 comprises a V_{H} sequence comprising a CDR-H1 sequence comprising SEQ ID NO: 10 or SEQ ID NO: 20, a CDR-H2 sequence comprising SEQ ID NO: 30 or SEQ ID NO: 40, and a CDR-H3 sequence comprising SEQ ID NO: 50.

### V_{H} Sequences

In some embodiments, the antibody that binds HLA-G comprises a V_{H} sequence comprising, consisting of, or consisting essentially of SEQ ID NO: 81. In some embodiments, the antibody that binds EGFR comprises a V_{H} sequence comprising, consisting of, or consisting essentially of SEQ ID NO: 89. In some embodiments, the antibody that binds PD-1 comprises a V_{H} sequence comprising, consisting of, or consisting essentially of SEQ ID NO: 82. In some embodiments, the antibody that binds PD-1 comprises a V_{H} sequence comprising, consisting of, or consisting essentially of SEQ ID NO: 83. In some embodiments, the antibody that binds PD-1 comprises a V_{H} sequence comprising, consisting of, or consisting essentially of SEQ ID NO: 84. In some embodiments, the antibody that binds PD-1 comprises a V_{H} sequence comprising, consisting of, or consisting essentially of SEQ ID NO: 85.

In some embodiments, the antibody that binds PD-L1 comprises a V_{H} sequence comprising, consisting of, or consisting essentially of SEQ ID NO: 86. In some embodiments, the antibody that binds PD-L1 comprises a V_{H} sequence comprising, consisting of, or consisting essentially of SEQ ID NO: 87. In some embodiments, the antibody that binds PD-L1 comprises a V_{H} sequence comprising, consisting of, or consisting essentially of SEQ ID NO: 88. In some embodiments, the antibody that binds CD47 comprises a V_{H} sequence comprising, consisting of, or consisting essentially of SEQ ID NO: 90.

### CDR-L1+CDR-L2+CDR-L3 Regions of the Antibody

In some embodiments, the antibody which binds to HLA-G comprises a V_{L} sequence comprising a CDR-L1 sequence comprising, consisting of, or consisting essentially of a SEQ ID NO: 51, a CDR-L2 sequence comprising, consisting of, or consisting essentially of a sequence selected from SEQ ID NO: 61, and a CDR-L3 sequence comprising, consisting of, or consisting essentially of a sequence selected from SEQ ID NO: 71. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{L} sequence having SEQ ID NO: 91.

In some embodiments, the antibody which binds to EGFR comprises a V_{L} sequence comprising a CDR-L1 sequence comprising, consisting of, or consisting essentially of SEQ ID NO: 59, a CDR-L2 sequence comprising, consisting of, or consisting essentially of SEQ ID NO: 69, and a CDR-L3 sequence comprising, consisting of, or consisting essentially of SEQ ID NO: 79. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{L} sequence having SEQ ID NO: 99.

In some embodiments, the antibody which binds to PD-1 comprises a V_{L} sequence comprising a CDR-L1 sequence comprising, consisting of, or consisting essentially of a sequence selected from any one of SEQ ID NOs: 52-55, a CDR-L2 sequence comprising, consisting of, or consisting essentially of a sequence selected from SEQ ID NOs: 62-65, and a CDR-L3 sequence comprising, consisting of, or consisting essentially of a sequence selected from SEQ ID NOs: SEQ ID NOs: 72-75. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{H} sequence selected from SEQ ID NOs: 92-95.

In some embodiments, the antibody that binds to PD-1 comprises a V_{L} sequence comprising a CDR-L1 sequence comprising, consisting of, or consisting essentially of a sequence comprising SEQ ID NO: 52, a CDR-L2 sequence comprising, consisting of, or consisting essentially of a sequence comprising SEQ ID NO: 62, and a CDR-L3 sequence comprising, consisting of, or consisting essentially of a sequence comprising SEQ ID NO: 72. In some embodiments, the antibody that binds to PD-1 comprises a V_{L} sequence comprising a CDR-L1 sequence comprising, consisting of, or consisting essentially of a sequence comprising SEQ ID NO: 53, a CDR-L2 sequence comprising, consisting of, or consisting essentially of a sequence comprising SEQ ID NO: 63, and a CDR-L3 sequence comprising, consisting of, or consisting essentially of a sequence comprising SEQ ID NO: 73. In some embodiments, the antibody that binds to PD-1 comprises a V_{L} sequence comprising a CDR-L1 sequence comprising, consisting of, or consisting essentially of a sequence comprising SEQ ID NO: 54, a CDR-L2 sequence comprising, consisting of, or consisting essentially of a sequence comprising SEQ ID NO: 64, and a CDR-L3 sequence comprising, consisting of, or consisting essentially of a sequence comprising SEQ ID NO: 74. In some embodiments, the antibody that binds to PD-1 comprises a V_{L} sequence comprising a CDR-L1 sequence comprising, consisting of, or consisting essentially of a sequence comprising SEQ ID NO: 55, a CDR-L2 sequence comprising, consisting of, or consisting essentially of a sequence comprising SEQ ID NO: 65, and a CDR-L3 sequence comprising, consisting of, or consisting essentially of a sequence comprising SEQ ID NO: 75.

In some embodiments, the CDR-L1 sequence, CDR-L2 sequence, and the CDR-L3 sequence are all from a single illustrative V_{L} sequence provided in this disclosure. For example, in some embodiments, the CDR-L1, CDR-L2, and CDR-L3 are all from a single V_{L} sequence having SEQ ID NO: 92. In some embodiments, the CDR-H1, CDR-H2, and CDR-H3 are all from a single V_{L} sequence having SEQ ID NO: 93. In some embodiments, the CDR-H1, CDR-H2, and CDR-H3 are all from a single V_{L} sequence having SEQ ID NO: 94. In some embodiments, the CDR-H1, CDR-H2, and CDR-H3 are all from a single V_{L} sequence having SEQ ID NO: 95.

In some embodiments, the antibody which binds to PD-L1 comprises a V_{L} sequence comprising a CDR-L1 sequence comprising, consisting of, or consisting essentially of a sequence selected from any one of SEQ ID NOs: 56-58, a CDR-L2 sequence comprising, consisting of, or consisting essentially of a sequence selected from SEQ ID NOs: 66-68, and a CDR-L3 sequence comprising, consisting of, or consisting essentially of a sequence selected from SEQ ID NOs: SEQ ID NOs: 76-78. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{H} sequence selected from SEQ ID NOs: 96-98.

In some embodiments, the antibody that binds to PD-L1 comprises a V_{L} sequence comprising a CDR-L1 sequence comprising, consisting of, or consisting essentially of a sequence comprising SEQ ID NO: 56, a CDR-L2 sequence comprising, consisting of, or consisting essentially of a sequence comprising SEQ ID NO: 66, and a CDR-L3 sequence comprising, consisting of, or consisting essentially of a sequence comprising SEQ ID NO: 76. In some embodiments, the antibody that binds to PD-L1 comprises a V_{L} sequence comprising a CDR-L1 sequence comprising, consisting of, or consisting essentially of a sequence comprising SEQ ID NO: 57, a CDR-L2 sequence comprising, consisting of, or consisting essentially of a sequence comprising SEQ ID NO: 67, and a CDR-L3 sequence comprising, consisting of, or consisting essentially of a sequence comprising SEQ ID NO: 77. In some embodiments, the antibody that binds to PD-L1 comprises a V_{L} sequence comprising a CDR-L1 sequence comprising, consisting of, or consisting essentially of a sequence comprising SEQ ID NO: 58, a CDR-L2 sequence comprising, consisting of, or consisting essentially of a sequence comprising SEQ ID NO: 68, and a CDR-L3 sequence comprising, consisting of, or consisting essentially of a sequence comprising SEQ ID NO: 78.

In some embodiments, the CDR-L1 sequence, CDR-L2 sequence, and the CDR-L3 sequence are all from a single illustrative V_{L} sequence provided in this disclosure. For example, in some embodiments, the CDR-L1, CDR-L2, and CDR-L3 are all from a single V_{L} sequence having SEQ ID NO: 96. In some embodiments, the CDR-H1, CDR-H2, and CDR-H3 are all from a single V_{L} sequence having SEQ ID NO: 97. In some embodiments, the CDR-H1, CDR-H2, and CDR-H3 are all from a single V_{L} sequence having SEQ ID NO: 98.

In some embodiments, the antibody which binds to CD47 comprises a V_{L} sequence comprising a CDR-L1 sequence comprising, consisting of, or consisting essentially of SEQ ID NO: 60, a CDR-L2 sequence comprising, consisting of, or consisting essentially of a sequence selected from SEQ ID NO: 70, and a CDR-L3 sequence comprising, consisting of, or consisting essentially of a sequence selected from SEQ ID NO: 80. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{L} sequence having SEQ ID MO: 100.

### V_{L} Sequences

In some embodiments, the antibody that binds to HLA-G comprises a V_{L} sequence comprising, consisting of, or consisting essentially of sequence SEQ ID NO: 91. In some embodiments, the antibody that binds to EGFR comprises a V_{L} sequence comprising, consisting of, or consisting essentially of sequence SEQ ID NO: 99. In some embodiments, the antibody that binds to PD-1 comprises a V_{L} sequence comprising, consisting of, or consisting essentially of sequence SEQ ID NO: 92. In some embodiments, the antibody that binds to PD-1 comprises a V_{L} sequence comprising, consisting of, or consisting essentially of sequence SEQ ID NO: 93. In some embodiments, the antibody that binds to PD-1 comprises a V_{L} sequence comprising, consisting of, or consisting essentially of sequence SEQ ID NO: 94. In some embodiments, the antibody that binds to PD-1 comprises a V_{L} sequence comprising, consisting of, or consisting essentially of sequence SEQ ID NO: 95.

In some embodiments, the antibody that binds to PD-L1 comprises a V_{L} sequence comprising, consisting of, or consisting essentially of sequence SEQ ID NO: 96. In some embodiments, the antibody that binds to PD-L1 comprises a V_{L} sequence comprising, consisting of, or consisting essentially of sequence SEQ ID NO: 97. In some embodiments, the antibody that binds to PD-1 comprises a V_{L} sequence comprising, consisting of, or consisting essentially of sequence SEQ ID NO: 98. In some embodiments, the antibody that binds to CD47 comprises a V_{L} sequence comprising, consisting of, or consisting essentially of SEQ ID NO: 100.

V_{H}-V_{L} Pairs

In some embodiments, the antibody which binds HLA-G comprises a V_{H} sequence and a V_{L} sequence, the V_{H} sequence comprising, consisting of, or consisting essentially of SEQ ID NO: 81 and the V_{L} sequence comprising, consisting of, or consisting essentially of SEQ ID NO: 91. In some embodiments, the antibody which binds EGFR comprises a V_{H} sequence and a V_{L} sequence, the V_{H} sequence comprising, consisting of, or consisting essentially of SEQ ID NO: 89 and the V_{L} sequence comprising, consisting of, or consisting essentially of SEQ ID NO: 99. In some embodiments, the antibody which binds CD47 comprises a V_{H} sequence and a V_{L} sequence, the V_{H} sequence comprising, consisting of, or consisting essentially of SEQ ID NO: 90 and the V_{L} sequence comprising, consisting of, or consisting essentially of SEQ ID NO: 100.

In some embodiments, the antibody which binds PD-1 comprises a V_{H} sequence and a V_{L} sequence, the V_{H} sequence comprising, consisting of, or consisting essentially of a sequence selected from any one of SEQ ID NOs: 82-85 and the V_{L} sequence comprising, consisting of, or consisting essentially a sequence selected from any one of SEQ ID NOs: 92-95. In some embodiments, the antibody which binds PD-1 comprises a V_{H} sequence and a V_{L} sequence, the V_{H} sequence comprising, consisting of, or consisting essentially of SEQ ID NO: 82 and the V_{L} sequence comprising, consisting of, or consisting essentially of SEQ ID NO: 92. In some embodiments, the antibody which binds PD-1 comprises a V_{H} sequence and a V_{L} sequence, the V_{H} sequence comprising, consisting of, or consisting essentially of SEQ ID NO: 83 and the V_{L} sequence comprising, consisting of, or consisting essentially of SEQ ID NO: 93. In some embodiments, the antibody which binds PD-1 comprises a V_{H} sequence and a V_{L} sequence, the V_{H} sequence comprising, consisting of, or consisting essentially of SEQ ID NO: 84 and the V_{L} sequence comprising, consisting of, or consisting essentially of SEQ ID NO: 94. In some embodiments, the antibody which binds PD-1 comprises a V_{H} sequence and a V_{L} sequence, the V_{H} sequence comprising, consisting of, or consisting essentially of SEQ ID NO: 85 and the V_{L} sequence comprising, consisting of, or consisting essentially of SEQ ID NO: 95.

In some embodiments, the antibody which binds PD-L1 comprises a V_{H} sequence and a V_{L} sequence, the V_{H} sequence comprising, consisting of, or consisting essentially of a sequence selected from any one of SEQ ID NOs: 86-88 and the V_{L} sequence comprising, consisting of, or consisting essentially a sequence selected from any one of SEQ ID NOs: 96-98. In some embodiments, the antibody which binds PD-L1 comprises a V_{H} sequence and a V_{L} sequence, the V_{H} sequence comprising, consisting of, or consisting essentially of SEQ ID NO: 86 and the V_{L} sequence comprising, consisting of, or consisting essentially of SEQ ID NO: 96. In some embodiments, the antibody which binds PD-L1 comprises a V_{H} sequence and a V_{L} sequence, the V_{H} sequence comprising, consisting of, or consisting essentially of SEQ ID NO: 87 and the V_{L} sequence comprising, consisting of, or consisting essentially of SEQ ID NO: 97. In some embodiments, the antibody which binds PD-L1 comprises a V_{H} sequence and a V_{L} sequence, the V_{H} sequence comprising, consisting of, or consisting essentially of SEQ ID NO: 88 and the V_{L} sequence comprising, consisting of, or consisting essentially of SEQ ID NO: 99.

### CDR-H1+CDR-H2+CDR-H3+CDR-L1+CDR-L2+CDR-L3

In some embodiments, the antibody which binds to HLA-G comprises or consists of a heavy chain variable region (V_{H}) and a light chain variable region (V_{L}), with V_{H} and/or V_{L} comprising:
a) a VHCDR1 having the sequence set forth in SEQ ID NO: 1 or SEQ ID NO: 11,
b) a VHCDR2 having the sequence set forth in SEQ ID NO: 21 or SEQ ID NO: 31,
c) a VHCDR3 having the sequence set forth in SEQ ID NO: 41,
d) a VLCDR1 having the sequence set forth in SEQ ID NO: 51,
e) a VLCDR2 having the sequence set forth in SEQ ID NO: 61, and
f) a VLCDR3 having the sequence set forth in SEQ ID NO: 71.

In some embodiments, the antibody which binds to EGFR comprises or consists of a heavy chain variable region (V_{H}) and a light chain variable region (V_{L}), with V_{H} and/or V_{L} comprising:
a) a VHCDR1 having the sequence set forth in SEQ ID NO: 9 or SEQ ID NO: 19,
b) a VHCDR2 having the sequence set forth in SEQ ID NO: 29 or SEQ ID NO: 39,
c) a VHCDR3 having the sequence set forth in SEQ ID NO: 49,
d) a VLCDR1 having the sequence set forth in SEQ ID NO: 59,
e) a VLCDR2 having the sequence set forth in SEQ ID NO: 69, and
f) a VLCDR3 having the sequence set forth in SEQ ID NO: 79.

In some embodiments, the antibody which binds to PD-1 comprises or consists of a heavy chain variable region (V_{H}) and a light chain variable region (V_{L}), with V_{H} and/or V_{L} comprising:
a) a VHCDR1 having the sequence set forth in any one of SEQ ID NOs: 2-5 or SEQ ID NOs: 12-15,
b) a VHCDR2 having the sequence set forth in any one of SEQ ID NOs: 22-25 or SEQ ID NOs: 32-35,
c) a VHCDR3 having the sequence set forth in any one of SEQ ID NOs: 42-45,
d) a VLCDR1 having the sequence set forth in any one of SEQ ID NOs: 52-55,
e) a VLCDR2 having the sequence set forth in any one of SEQ ID NOs: 62-65, and
f) a VLCDR3 having the sequence set forth in any one of SEQ ID NOs: 72-75.

In some embodiments, the antibody which binds to PD-1 comprises a V_{H} sequence comprising a CDR-H1 sequence comprising SEQ ID NO: 2 or SEQ ID NO: 12, a CDR-H2 sequence comprising SEQ ID NO: 22 or SEQ ID NO: 32, and a CDR-H3 sequence comprising SEQ ID NO: 42 and a VL sequence comprising a CDR-L1 sequence comprising SEQ ID NO: 52, a CDR-L2 sequence comprising SEQ ID NO: 62, and a CDR-L3 sequence SEQ ID NO: 72. In some embodiments, the antibody which binds to PD-1 comprises a V_{H} sequence comprising a CDR-H1 sequence comprising SEQ ID NO: 3 or SEQ ID NO: 13, a CDR-H2 sequence comprising SEQ ID NO: 23 or SEQ ID NO: 33, and a CDR-H3 sequence comprising SEQ ID NO: 43 and a VL sequence comprising a CDR-L1 sequence comprising SEQ ID NO: 53, a CDR-L2 sequence comprising SEQ ID NO: 63, and a CDR-L3 sequence SEQ ID NO: 73. In some embodiments, the antibody which binds to PD-1 comprises a V_{H} sequence comprising a CDR-H1 sequence comprising SEQ ID NO: 4 or SEQ ID NO: 14, a CDR-H2 sequence comprising SEQ ID NO: 24 or SEQ ID NO: 34, and a CDR-H3 sequence comprising SEQ ID NO: 44 and a VL sequence comprising a CDR-L1 sequence comprising SEQ ID NO: 54, a CDR-L2 sequence comprising SEQ ID NO: 64, and a CDR-L3 sequence SEQ ID NO: 74. In some embodiments, the antibody which binds to PD-1 comprises a V_{H} sequence comprising a CDR-H1 sequence comprising SEQ ID NO: 5 or SEQ ID NO: 15, a CDR-H2 sequence comprising SEQ ID NO: 25 or SEQ ID NO: 35, and a CDR-H3 sequence comprising SEQ ID NO: 45 and a VL sequence comprising a CDR-L1 sequence comprising SEQ ID NO: 55, a CDR-L2 sequence comprising SEQ ID NO: 65, and a CDR-L3 sequence SEQ ID NO: 75.

In some embodiments, the antibody which binds to PD-L1 comprises or consists of a heavy chain variable region (V_{H}) and a light chain variable region (V_{L}), with V_{H} and/or V_{L} comprising:
a) a VHCDR1 having the sequence set forth in any one of SEQ ID NOs: 6-8 or SEQ ID NOs: 16-18,
b) a VHCDR2 having the sequence set forth in any one of SEQ ID NOs: 26-28 or SEQ ID NOs: 38-38,
c) a VHCDR3 having the sequence set forth in any one of SEQ ID NOs: 46-48,
d) a VLCDR1 having the sequence set forth in any one of SEQ ID NOs: 56-58,
e) a VLCDR2 having the sequence set forth in any one of SEQ ID NOs: 66-68, and
f) a VLCDR3 having the sequence set forth in any one of SEQ ID NOs: 76-78.
   In some embodiments, the antibody which binds to PD-L1 comprises a V_{H} sequence comprising a CDR-H1 sequence comprising SEQ ID NO: 6 or SEQ ID NO: 16, a CDR-H2 sequence comprising SEQ ID NO: 26 or SEQ ID NO: 36, and a CDR-H3 sequence comprising SEQ ID NO: 46 and a VL sequence comprising a CDR-L1 sequence comprising SEQ ID NO: 56, a CDR-L2 sequence comprising SEQ ID NO: 66, and a CDR-L3 sequence SEQ ID NO: 76. In some embodiments, the antibody which binds to PD-L1 comprises a V_{H} sequence comprising a CDR-H1 sequence comprising SEQ ID NO: 7 or SEQ ID NO: 17, a CDR-H2 sequence comprising SEQ ID NO: 27 or SEQ ID NO: 37, and a CDR-H3 sequence comprising SEQ ID NO: 47 and a VL sequence comprising a CDR-L1 sequence comprising SEQ ID NO: 57, a CDR-L2 sequence comprising SEQ ID NO: 67, and a CDR-L3 sequence SEQ ID NO: 77. In some embodiments, the antibody which binds to PD-L1 comprises a V_{H} sequence comprising a CDR-H1 sequence comprising SEQ ID NO: 8 or SEQ ID NO: 18, a CDR-H2 sequence comprising SEQ ID NO: 28 or SEQ ID NO: 38, and a CDR-H3 sequence comprising SEQ ID NO: 48 and a VL sequence comprising a CDR-L1 sequence comprising SEQ ID NO: 58, a CDR-L2 sequence comprising SEQ ID NO: 68, and a CDR-L3 sequence SEQ ID NO: 78.

In some embodiments, the antibody which binds to CD47 comprises or consists of a heavy chain variable region (V_{H}) and a light chain variable region (V_{L}), with V_{H} and/or V_{L} comprising:
a) a VHCDR1 having the sequence set forth in SEQ ID NO: 10 or SEQ ID NO: 20,
b) a VHCDR2 having the sequence set forth in SEQ ID NO: 30 or SEQ ID NO: 40,
c) a VHCDR3 having the sequence set forth in SEQ ID NO: 50,
d) a VLCDR1 having the sequence set forth in SEQ ID NO: 60,
e) a VLCDR2 having the sequence set forth in SEQ ID NO: 70, and
f) a VLCDR3 having the sequence set forth in SEQ ID NO: 80.

### HC+LC

In some embodiments, the antibody that binds HLA-G, EGFR, PD-1, PD-L1, and/or CD47 comprises or consists of one or more heavy chains consisting of an HC sequence and one or more light chains consisting of an LC sequence. In some embodiments, the antibody that binds HLA-G, EGFR, PD-1, PD-L1, and/or CD47 or SIRPα comprises or consists of two identical heavy chains comprising, consisting of, or consisting essentially of an HC sequence and two identical light chains comprising, consisting of, or consisting essentially of an LC sequence.

In some embodiments, the HC sequence of the antibody that binds HLA-G is an HC sequence comprising, consisting of, or consisting essentially of SEQ ID NO: 101 and the LC sequence of the antibody that binds HLA-G is an LC sequence comprising, consisting of, or consisting essentially of any one of SEQ ID NO: 111. In some embodiments, the HC sequence of the antibody that binds EGFR is an HIC sequence comprising, consisting of, or consisting essentially of SEQ ID NO: 109 and the LC sequence of the antibody that binds EGFR is an LC sequence comprising, consisting of, or consisting essentially of SEQ ID NO: 119. In some embodiments, the HC sequence of the antibody that binds CD47 is an HC sequence comprising, consisting of, or consisting essentially of SEQ ID NO: 110 and the LC sequence of the antibody that binds PD-1 is an LC sequence comprising, consisting of, or consisting essentially of SEQ ID NO: 120.

In some embodiments, the HC sequence of the antibody that binds PD-1 is an HC sequence comprising, consisting of, or consisting essentially of any one of SEQ ID NOs: 102-105 and the LC sequence of the antibody that binds PD-1 is an LC sequence comprising, consisting of, or consisting essentially any one of SEQ ID NOs: 112-115. In some embodiments, the HC sequence of the antibody that binds PD-1 is an HC sequence comprising, consisting of, or consisting essentially of SEQ ID NO: 102 and the LC sequence of the antibody that binds PD-1 is an LC sequence comprising, consisting of, or consisting essentially of SEQ ID NO: 112. In some embodiments, the HC sequence of the antibody that binds PD-1 is an HC sequence comprising, consisting of, or consisting essentially of SEQ ID NO: 103 and the LC sequence of the antibody that binds PD-1 is an LC sequence comprising, consisting of, or consisting essentially of SEQ ID NO: 113. In some embodiments, the HC sequence of the antibody that binds PD-1 is an HC sequence comprising, consisting of, or consisting essentially of SEQ ID NO: 104 and the LC sequence of the antibody that binds PD-1 is an LC sequence comprising, consisting of, or consisting essentially of SEQ ID NO: 114. In some embodiments, the HC sequence of the antibody that binds PD-1 is an HC sequence comprising, consisting of, or consisting essentially of SEQ ID NO: 105 and the LC sequence of the antibody that binds PD-1 is an LC sequence comprising, consisting of, or consisting essentially of SEQ ID NO: 115.

In some embodiments, the HC sequence of the antibody that binds PD-L1 is an HC sequence comprising, consisting of, or consisting essentially of any one of SEQ ID NOs: 106-108 and the LC sequence of the antibody that binds PD-1 is an LC sequence comprising, consisting of, or consisting essentially any one of SEQ ID NOs: 118-118. In some embodiments, the HC sequence of the antibody that binds PD-L1 is an HC sequence comprising, consisting of, or consisting essentially of SEQ ID NO: 106 and the LC sequence of the antibody that binds PD-L1 is an LC sequence comprising, consisting of, or consisting essentially of SEQ ID NO: 116. In some embodiments, the HC sequence of the antibody that binds PD-L1 is an HC sequence comprising, consisting of, or consisting essentially of SEQ ID NO: 107 and the LC sequence of the antibody that binds PD-L1 is an LC sequence comprising, consisting of, or consisting essentially of SEQ ID NO: 117. In some embodiments, the HC sequence of the antibody that binds PD-L1 is an HC sequence comprising, consisting of, or consisting essentially of SEQ ID NO: 118 and the LC sequence of the antibody that binds PD-L1 is an LC sequence comprising, consisting of, or consisting essentially of SEQ ID NO: 118.

In some embodiments, the HC sequence of the antibody that binds HLA-G is an HC sequence comprising or consisting of SEQ ID NO: 101 and the LC sequence is an LC sequence comprising or consisting of SEQ ID NO: 111 and the HC sequence of the antibody that binds PD-1 is an HC sequence comprising or consisting of SEQ ID NO: 102 and the LC sequence is an LC sequence comprising or consisting of SEQ ID NO: 112. In some embodiments, the HC sequence of the antibody that binds HLA-G is an HC sequence comprising or consisting of SEQ ID NO: 101 and the LC sequence is an LC sequence comprising or consisting of SEQ ID NO: 111 and the HC sequence of the antibody that binds PD-1 is an HC sequence comprising or consisting of SEQ ID NO: 103 and the LC sequence is an LC sequence comprising or consisting of SEQ ID NO: 113. In some embodiments, the HC sequence of the antibody that binds HLA-G is an HC sequence comprising or consisting of SEQ ID NO: 101 and the LC sequence is an LC sequence comprising or consisting of SEQ ID NO: 111 and the HC sequence of the antibody that binds PD-1 is an HC sequence comprising or consisting of SEQ ID NO: 104 and the LC sequence is an LC sequence comprising or consisting of SEQ ID NO: 114. In some embodiments, the HC sequence of the antibody that binds HLA-G is an HC sequence comprising or consisting of SEQ ID NO: 101 and the LC sequence is an LC sequence comprising or consisting of SEQ ID NO: 111 and the HC sequence of the antibody that binds PD-1 is an HC sequence comprising or consisting of SEQ ID NO: 105 and the LC sequence is an LC sequence comprising or consisting of SEQ ID NO: 115.

In some embodiments, the HC sequence of the antibody that binds HLA-G is an HC sequence comprising or consisting of SEQ ID NO: 101 and the LC sequence is an LC sequence comprising or consisting of SEQ ID NO: 111 and the HC sequence of the antibody that binds PD-L1 is an HC sequence comprising or consisting of SEQ ID NO: 106 and the LC sequence is an LC sequence comprising or consisting of SEQ ID NO: 116. In some embodiments, the HC sequence of the antibody that binds HLA-G is an HC sequence comprising or consisting of SEQ ID NO: 101 and the LC sequence is an LC sequence comprising or consisting of SEQ ID NO: 111 and the HC sequence of the antibody that binds PD-L1 is an HC sequence comprising or consisting of SEQ ID NO: 107 and the LC sequence is an LC sequence comprising or consisting of SEQ ID NO: 117. In some embodiments, the HC sequence of the antibody that binds HLA-G is an HC sequence comprising or consisting of SEQ ID NO: 101 and the LC sequence is an LC sequence comprising or consisting of SEQ ID NO: 111 and the HC sequence of the antibody that binds PD-L1 is an HC sequence comprising or consisting of SEQ ID NO: 108 and the LC sequence is an LC sequence comprising or consisting of SEQ ID NO: 118.

In some embodiments, the HC sequence of the antibody that binds HLA-G is an HC sequence comprising or consisting of SEQ ID NO: 101 and the LC sequence is an LC sequence comprising or consisting of SEQ ID NO. 111 and the HC sequence of the antibody that binds EGFR is an HC sequence comprising or consisting of SEQ ID NO: 109 and the LC sequence is an LC sequence comprising or consisting of SEQ ID NO: 119. In some embodiments, the HC sequence of the antibody that binds HLA-G is an HC sequence comprising or consisting of SEQ ID NO: 101 and the LC sequence is an LC sequence comprising or consisting of SEQ ID NO: 111 and the HC sequence of the antibody that binds CD47 is an HC sequence comprising or consisting of SEQ ID NO: 110 and the LC sequence is an LC sequence comprising or consisting of SEQ ID NO: 120.

### Glycosylation Variants

In certain embodiments, an antibody of the invention may be altered to increase, decrease, or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

In certain embodiments, the antibody is glycosylated. In certain embodiments, the antibody is deglycosylated. Carbohydrates may be removed by standard techniques. In certain embodiments, the antibody is aglycosylated, for instance by expression in a system that does not glycosylate.

### Preparation of Antibodies

HLA-G, PD-1, PD-L1, and/or CD47 or SIRPα antigens may used for production of antibodies. They may be intact proteins or fragments of the antigens. They may be in the form of an isolated protein or expressed by a cell. Other forms of antigens useful for generating antibodies will be apparent to those skilled in the art.

DNA encoding the antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies. Other ways may be useful for generating antibodies will be apparent to those skilled in the art.

### Cancers

For cancers, the pharmaceutical compositions of the invention are generally administered to a human or other organism or animal in a pharmaceutically acceptable dosage form. Any suitable cancer may be treated with the antibodies provided herein. In some embodiments, the cancer is a hematological cancer. In some embodiments, the cancer is a solid cancer.

In some embodiments, the compositions and methods increase macrophage phagocytosis. In some embodiments, the compositions and methods enhance ILT2⁺CD8⁺ T cell degranulation (CD107a). In some embodiments, increased macrophage phagocytosis is of lung cancer cells. In some embodiments, the lung cancer cells are HLA-G⁺EGFR⁺ lung cancer cells. In some embodiments, increased macrophage phagocytosis is of HLA-G⁺ A549, HLA-G⁺ SK-OV-3, HT-1376, and/or JEG-3 cells.

### EXAMPLES

### Example 1: Combination of the Antibody that Binds HLA-G (Fab Fragment) and Cetuximab Increased Macrophage Phagocytosis of HLA-G⁺ EGFR⁺ Lung Cancer Cells Above Either Single Agent Alone

To determine the potential clinical benefit of the antibody that binds HLA-G in combination with cetuximab, a Fab fragment of the antibody that binds HLA-G was evaluated in a macrophage assay where cetuximab was used to induce phagocytosis of EGFR⁺ A549 lung cancer cells engineered to express HLA-G.

CD14⁺ enriched cells were differentiated into adherent macrophages by incubating at 37° C., 5% CO₂ for 7 days in complete RPMI (cRPMI) with recombinant human M-CSF Cells were harvested after 7 days, washed, and resuspended in cRPMI. Cells were plated in a 96-well round bottom plate at 50,000 cells/well in 50 μl cRPMI and incubated at 37° C., 5% CO₂, before being combined with the target cells.

The target cells (A549 lung cancer cells engineered to express HLA-G) were stained with CellTrace™ Violet (CTV, 1:1000) at 37° C. in PBS, washed, and plated in cRPMI at 25,000 cells per well. Cells were subsequently incubated with antibodies (the antibody that binds HLA-G Fab and/or cetuximab) for 1 hour at 37° C., 5% CO₂. The mixture of A549 cells and antibodies was then combined with macrophages and incubated for 2 hours at 37° C., 5% CO₂. After the 2-hour incubation, cells were blocked with wash buffer/BD Fc Block™/6% mouse serum for 20 minutes and then stained with anti-CD11b antibody and live/dead discrimination dye for 20 minutes. Cells were then washed, resuspended in wash buffer, and analyzed on the BD Fortessa flow cytometer.

Sample data was exported as FCS files and analyzed using FlowJo software v10. Macrophages were evaluated for the presence of CTV as a readout of phagocytic uptake of CTV⁺ target cells. Live cells were gated on CD11b⁺/CTV⁺ and reported as a percent of total live macrophages.

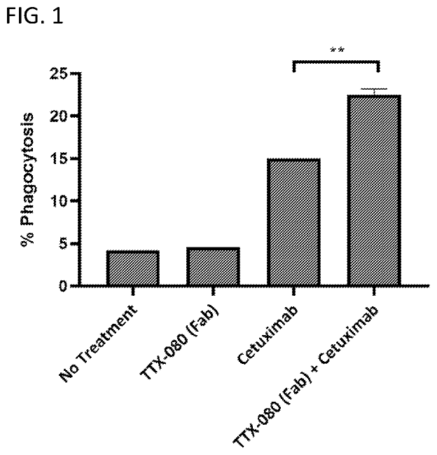

FIG. 1 shows that the combination of the antibody that binds HLA-G Fab with cetuximab significantly increased target cell phagocytosis compared to cetuximab alone. No activity was observed by an antibody that binds HLA-G Fab compared to an untreated control.

### Example 2: The Antibody that Binds HLA-G in Combination with Pembrolizumab or Atezolizumab Enhanced ILT2⁺ CD8⁺ T Cell Degranulation (CD107a) Above Either Single Agent Alone

To demonstrate that an anti-PD-1 antibody (pembrolizumab) or an anti-PD-L1 antibody (atezolizumab) can be used in combination with the anti-HLA-G antibody to enhance human T cell functional activity, 721.221 PD-L1⁺ HLA-G⁺ cells were co-cultured with human CD8⁺ T cells.

Isolated CD8⁺ T cells were plated in cRPMI in a 96-well round bottom plate at 50,000 cells/well. The CD8⁺ T cells were subsequently incubated with ImmunoCult Human CD3/CD28 T cell activator in addition to pembrolizumab for 1 hour at 37° C., 5% CO₂. Target cells (721.221 engineered to express PD-L1 and HLA-G) were plated in cRPMI at 50,000 cells/well and subsequently incubated with the antibody that binds HLA-G or a combination of the antibody that binds HLA-G and atezolizumab for 1 hour at 37° C., 5% CO₂. The mixture of CD8⁺ T cells was then combined with the target cells and incubated for 24 hours at 37° C., 5% CO₂. The final ratio of CD8⁻ T cells to target cells was 1:1.

After the 24-hour incubation, a mixture containing a 1:100 final concentration of eFluor660 αCD107a antibody and 1:1000 final concentration of Monensin were added to the CD8⁺ T cell-target cell co-culture and the cells were incubated with the mixture for 4 hours at 37° C., 5% CO₂. Following the incubation, the cells were washed once in 4° C. wash buffer (Phosphate Buffered Saline, 2% FBS, 2 mM EDTA) and then cells were blocked with wash buffer/BD Fc Block™/6% mouse serum for 20 minutes. Staining solution was then added to the cells consisting of 1:2000 Fixable Viability Dye eFluor™ 780 (Invitrogen) and fluorescent-labeled αILT2, αCD8, and αPD-1 antibodies for 25 minutes at 4° C. Cells were washed again in 4° C. wash buffer before being resuspended in fixation/permeabilization buffer consisting of 1 part Fixation/Permeabilization concentrate and 3 parts Fixation/Permeabilization Diluent (Invitrogen) for 30 minutes at 4° C. The cells were then washed with Permeabilization Buffer (Invitrogen) before a final wash in 4° C. wash buffer and resuspension in wash buffer.

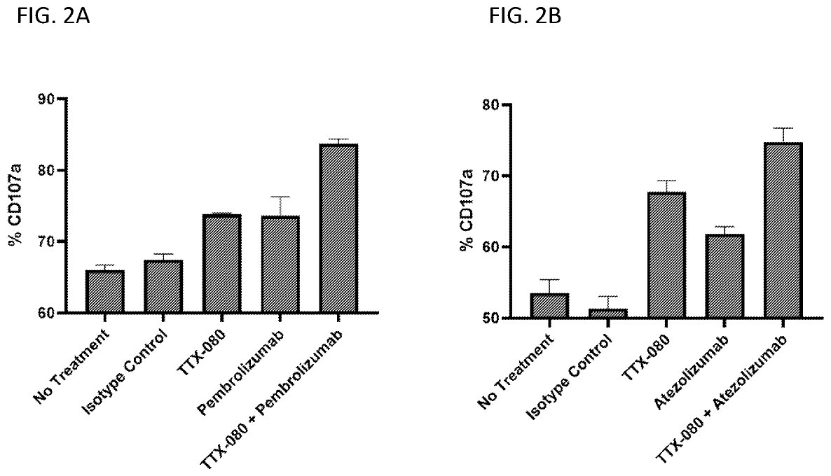

The cells were analyzed for percent expression of CD107a on the ILT2⁺ CD8⁺ T cells by flow cytometry analysis on a BD LSR Fortessa. As shown in FIGS. 2A and 2B, the combination of the antibody that binds HLA-G with pembrolizumab or atezolizumab, respectively, enhanced ILT2⁺ CD8⁺ T cell degranulation above either single agent alone.

### Example 3: Combination of the Antibody that Binds HLA-G and Magrolimab Increased Macrophage Phagocytosis of HLA-G⁺ A549, HLA-G⁺ SK-OV-3, HT-1376, and JEG-3 Target Cells Above Either Single Agent Alone. Triple-Agent Combination Showed Increased Activity Compared to Single or Double-Agent Treatment Groups

To determine the potential clinical benefit of the antibody that binds HLA-G in combination with magrolimab and/or cetuximab, agents were evaluated in a macrophage assay measuring phagocytosis of A549 lung cancer cells and SK-OV-3 ovarian cancer cells engineered to express HLA-G as well as HT-1376 bladder carcinoma and JEG-3 choriocarcinoma cells that endogenously express HLA-G.

CD14⁺ enriched cells were differentiated into adherent macrophages by incubating at 37° C., 5% CO₂ for 7 days in complete RPMI (cRPMI) with recombinant human M-CSF. Cells were harvested after 7 days, washed, and resuspended in cRPMI. Cells were plated in a 96-well round bottom plate at 100,000 macrophages per well in 50 μl cRPMI and incubated at 37° C., 5% CO₂, before being combined with the target cells (A549, SK-OV-3, HT-1376, JEG-3 cell lines)

The target cells were stained with CellTrace™ Violet (CTV, 1:1000) at 37° C. in PBS, washed, and plated in cRPMI at 50,000 target cells per well. Cells were subsequently incubated with antibodies (the antibody that binds HLA-G and/or magrolimab and/or cetuximab) for 1 hour at 37° C., 5% CO₂. The mixture of target cells and antibodies was then combined with macrophages and incubated for 2 hours at 37° C., 5% CO₂. After the 2-hour incubation, cells were blocked with wash buffer/BD Fc Block™/6% mouse serum for 20 minutes and then stained with anti-CD11b antibody and live/dead discrimination dye for 20 minutes. Cells were then washed, resuspended in wash buffer, and analyzed on the BD Fortessa flow cytometer.

Sample data was exported as FCS files and analyzed using FlowJo software v10. Macrophages were evaluated for the presence of CTV as a readout of phagocytic uptake of CTV⁺ target cells. Live cells were gated on CD11b/CTV and reported as a percent of total live macrophages.

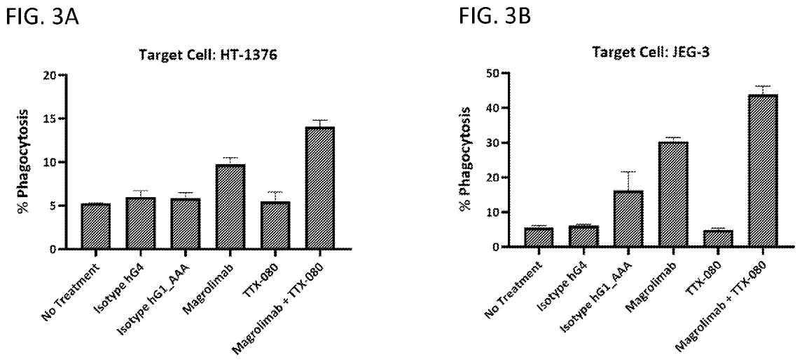

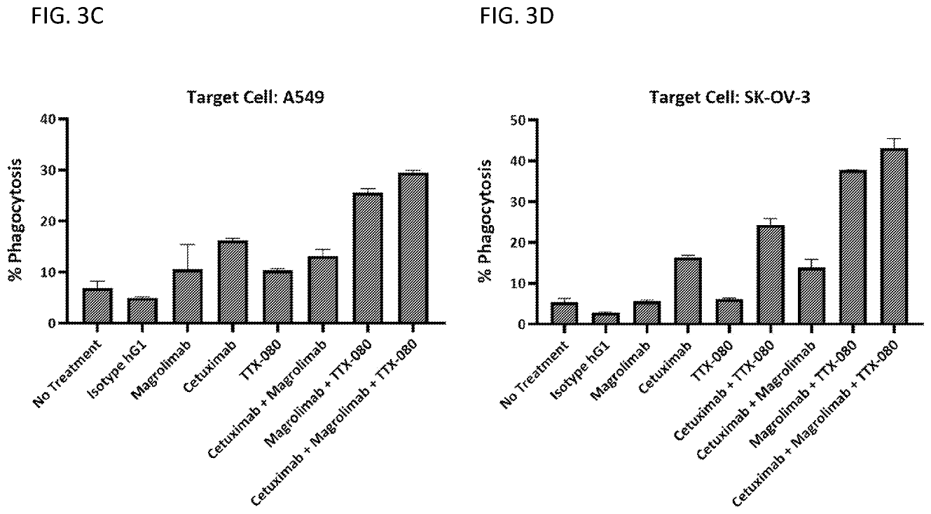

FIG. 3A and FIG. 3B demonstrate an antibody that binds HLA G in combination with magrolimab increased macrophage phagocytosis of endogenous HLA-G expressing HT1376 and JEG-3 cells, respectively, compared to either single agent alone. FIG. 3C and FIG. 3D show that a triple-agent combination of an antibody that binds HLA-G together with magrolimab and cetuximab has greater activity than single or double-agent treatments to induce phagocytosis of A549 and SK-OV-3 cells engineered to express HLA-G. Isotype antibodies were used as negative controls.

### Example S: Sequences

TABLE S
provides sequences referred to herein.

| SEQ | | | |
|---|---|---|---|
| ID | | | |
| NO: | Region | Antibody | Sequence |
| 1 | CDR-H1 | Anti-HLA-G | GYSILSGY |
| | (Chothia) | | |
| 2 | CDR-H1 | Nivolumab (PD-1) | GITFSNS |
| | (Chothia) | | |
| 3 | CDR-H1 | Pembrolizumab | GYTFTNY |
| | (Chothia) | (PD-1) | |
| 4 | CDR-H1 | Cemiplimab (PD- | GFTFSNF |
| | (Chothia) | 1) | |
| 5 | CDR-H1 | Dostarlimab (PD- | GFTFSSY |
| | (Chothia) | 1) | |
| 6 | CDR-H1 | Atezolizumab | GFTFSDS |
| | (Chothia) | (PD-L1) | |
| 7 | CDR-H1 | Avelumab (PD- | GFTFSSY |
| | (Chothia) | L1) | |
| 8 | CDR-H1 | Durvalumab (PD- | GFTFSRY |
| | (Chothia) | L1) | |
| 9 | CDR-H1 | Cetuximab | GFSLTNY |
| | (Chothia) | (EGFR) | |
| 10 | CDR-H1 | Magrolimab | GYTFTNY |
| | (Chothia) | (CD47) | |
| 11 | CDR-H1 | Anti-HLA-G | SGYYWF |
| | (Kabat) | | |
| 12 | CDR-H1 | Nivolumab (PD-1) | NSGMH |
| | (Kabat) | | |
| 13 | CDR-H1 | Pembrolizumab | NYYMY |
| | (Kabat) | (PD-1) | |
| 14 | CDR-H1 | Cemiplimab (PD- | NFGMT |
| | (Kabat) | 1) | |
| 15 | CDR-H1 | Dostarlimab (PD- | SYDMS |
| | (Kabat) | 1) | |
| 16 | CDR-H1 | Atezolizumab | DSWIH |
| | (Kabat) | (PD-L1) | |
| 17 | CDR-H1 | Avelumab (PD- | SYIMM |
| | (Kabat) | L1 | |
| 18 | CDR-H1 | Durvalumab (PD- | RYWMS |
| | (Kabat) | L1) | |
| 19 | CDR-H1 | Cetuximab | NYGVH |
| | (Kabat) | (EGFR) | |
| 20 | CDR-H1 | Magrolimab | NYNMH |
| | (Kabat) | (CD47) | |
| 21 | CDR-H2 | Anti-HLA-G | YHSGS |
| | (Chothia) | | |
| 22 | CDR-H2 | Nivolumab (PD-1) | WYDGSK |
| | (Chothia) | | |
| 23 | CDR-H2 | Pembrolizumab | NPSNGG |
| | (Chothia) | (PD-1) | |
| 24 | CDR-H2 | Cemiplimab (PD- | SGGGRD |
| | (Chothia) | 1) | |
| 25 | CDR-H2 | Dostarlimab (PD- | SGGGSY |
| | (Chothia) | 1) | |
| 26 | CDR-H2 | Atezolizumab | SPYGGS |
| | (Chothia) | (PD-L1) | |
| 27 | CDR-H2 | Avelumab (PD- | YPSGGI |
| | (Chothia) | L1) | |
| 28 | CDR-H2 | Durvalumab (PD- | KQDGSE |
| | (Chothia) | L1) | |
| 29 | CDR-H2 | Cetuximab | WSGGN |
| | (Chothia) | (EGFR) | |
| 30 | CDR-H2 | Magrolimab | YPGNDD |
| | (Chothia) | (CD47) | |
| 31 | CDR-H2 | Anti-HLA-G | GIYHSGSTAYNPSLKS |
| | (Kabat) | | |
| 32 | CDR-H2 | Nivolumab (PD-1) | VIWYDGSKRYYADSVKG |
| | (Kabat) | | |
| 33 | CDR-H2 | Pembrolizumab | GINPSNGGTNFNEKFKN |
| | (Kabat) | (PD-1) | |
| 34 | CDR-H2 | Cemiplimab (PD- | GISGGGRDTYFADSVKG |
| | (Kabat) | 1) | |
| 35 | CDR-H2 | Dostarlimab (PD- | TISGGGSYTYYQDSVKG |
| | (Kabat) | 1) | |
| 36 | CDR-H2 | Atezolizumab | WISPYGGSTYYADSVKG |
| | (Kabat) | (PD-L1) | |
| 37 | CDR-H2 | Avelumab (PD- | SIYPSGGITFYADTVKG |
| | (Kabat) | L1) | |
| 38 | CDR-H2 | Durvalumab (PD- | NIKQDGSEKYYVDSVKG |
| | (Kabat) | L1) | |
| 39 | CDR-H2 | Cetuximab | VIWSGGNTDYNTPFTS |
| | (Kabat) | (EGFR) | |
| 40 | CDR-H2 | Magrolimab | TIYPGNDDTSYNQKFKD |
| | (Kabat) | (CD47) | |
| 41 | CDR-H3 | Anti-HLA-G | GGEVTYSRGPLDV |
| 42 | CDR-H3 | Nivolumab (PD-1) | NDDY |
| 43 | CDR-H3 | Pembrolizumab | RDYRFDMGFDY |
| | | (PD-1) | |
| 44 | CDR-H3 | Cemiplimab (PD- | WGNIYFDY |
| | | 1) | |
| 45 | CDR-H3 | Dostarlimab (PD- | PYYAMDY |
| | | 1) | |
| 46 | CDR-H3 | Atezolizumab | RHWPGGFDY |
| | | (PD-L1) | |
| 47 | CDR-H3 | Avelumab (PD- | IKLGTVTTVDY |
| | | L1) | |
| 48 | CDR-H3 | Durvalumab (PD- | EGGWFGELAFDY |
| | | L1) | |
| 49 | CDR-H3 | Cetuximab | ALTYYDYEFAY |
| | | (EGFR) | |
| 50 | CDR-H3 | Magrolimab | GGYRAMDY |
| | | (CD47) | |
| 51 | CDR-L1 | Anti-HLA-G | RASNAVSSSYLA |
| 52 | CDR-L1 | Nivolumab (PD-1) | RASQSVSSYLA |
| 53 | CDR-L1 | Pembrolizumab | RASKGVSTSGYSYLH |
| | | (PD-1) | |
| 54 | CDR-L1 | Cemiplimab (PD- | RASLSINTFLN |
| | | 1) | |
| 55 | CDR-L1 | Dostarlimab (PD- | KASQDVGTAVA |
| | | 1) | |
| 56 | CDR-L1 | Atezolizumab | RASQDVSTAVA |
| | | (PD-L1) | |
| 57 | CDR-L1 | Avelumab (PD- | TGTSSDVGGYNYVS |
| | | L1) | |
| 58 | CDR-L1 | Durvalumab (PD- | RASQRVSSSYLA |
| | | L1) | |
| 59 | CDR-L1 | Cetuximab | RASQSIGTNIH |
| | | (EGFR) | |
| 60 | CDR-L1 | Magrolimab | RSSQSIVYSNGNTYLG |
| | | (CD47) | |
| 61 | CDR-L2 | Anti-HLA-G | GASYRAT |
| 62 | CDR-L2 | Nivolumab (PD-1) | DASNRAT |
| 63 | CDR-L2 | Pembrolizumab | LASYLES |
| | | (PD-1) | |
| 64 | CDR-L2 | Cemiplimab (PD- | AASSLHG |
| | | 1) | |
| 65 | CDR-L2 | Dostarlimab (PD- | WASTLHT |
| | | 1) | |
| 66 | CDR-L2 | Atezolizumab | SASFLYS |
| | | (PD-L1) | |
| 67 | CDR-L2 | Avelumab (PD- | DVSNRPS |
| | | L1) | |
| 68 | CDR-L2 | Durvalumab (PD- | DASSRAT |
| | | L1) | |
| 69 | CDR-L2 | Cetuximab | YASESIS |
| | | (EGFR) | |
| 70 | CDR-L2 | Magrolimab | KVSNRFS |
| | | (CD47) | |
| 71 | CDR-L3 | Anti-HLA-G | QQHSLYPPT |
| 72 | CDR-L3 | Nivolumab (PD-1) | QQSSNWPRT |
| 73 | CDR-L3 | Pembrolizumab | QHSRDLPLT |
| | | (PD-1) | |
| 74 | CDR-L3 | Cemiplimab (PD- | QQSSNTPFT |
| | | 1) | |
| 75 | CDR-L3 | Dostarlimab (PD- | QHYSSYPWT |
| | | 1) | |
| 76 | CDR-L3 | Atezolizumab | QQYLYHPAT |
| | | (PD-L1) | |
| 77 | CDR-L3 | Avelumab (PD- | SSYTSSSTRV |
| | | L1) | |
| 78 | CDR-L3 | Durvalumab (PD- | QQYGSLPWT |
| | | L1) | |
| 79 | CDR-L3 | Cetuximab | QQNNNWPTT |
| | | (EGFR) | |
| 80 | CDR-L3 | Magrolimab | FQGSHVPYT |
| | | (CD47) | |
| 81 | VH | Anti-HLA-G | QVQLQESGPGLVKPSETLSLTCAVSGYSILSGYYWF |
| | | | WIRQPPGKGLEWIGGIYHSGSTAYNPSLKSRVTISV |
| | | | DTSKNQFSLKLSSVTAADTAVYYCARGGEVTYSRGP |
| | | | LDVWGQGTTVTVSS |
| 82 | VH | Nivolumab (PD-1) | QVQLVESGGGVVQPGRSLRLDCKASGITFSNSGMHW |
| | | | VRQAPGKGLEWVAVIWYDGSKRYYADSVKGRFTISR |
| | | | DNSKNTLFLQMNSLRAEDTAVYYCATNDDYWGQGTL |
| | | | VTVSS |
| 83 | VH | Pembrolizumab | QVQLVQSGVEVKKPGASVKVSCKASGYTFTNYYMYW |
| | | (PD-1) | VRQAPGQGLEWMGGINPSNGGTNFNEKFKNRVTLTT |
| | | | DSSTTTAYMELKSLQFDDTAVYYCARRDYRFDMGFD |
| | | | YWGQGTTVTVSS |
| 84 | VH | Cemiplimab (PD- | EVQLLESGGVLVQPGGSLRLSCAASGFTFSNFGMTW |
| | | 1) | VRQAPGKGLEWVSGISGGGRDTYFADSVKGRFTISR |
| | | | DNSKNTLYLQMNSLKGEDTAVYYCVKWGNIYFDYWG |
| | | | QGTLVTVSS |
| 85 | VH | Dostarlimab (PD- | EVQLLESGGGLVQPGGSLRLSCAASGFTFSSYDMSW |
| | | 1) | VRQAPGKGLEWVSTISGGGSYTYYQDSVKGRFTISR |
| | | | DNSKNTLYLQMNSLRAEDTAVYYCASPYYAMDYWGQ |
| | | | GTTVTVSS |
| 86 | VH | Atezolizumab | EVQLVESGGGLVQPGGSLRLSCAASGFTFSDSWIHW |
| | | (PD-L1) | VRQAPGKGLEWVAWISPYGGSTYYADSVKGRFTISA |
| | | | DTSKNTAYLQMNSLRAEDTAVYYCARRHWPGGFDYW |
| | | | GQGTLVTVSS |
| 87 | VH | Avelumab (PD- | EVQLLESGGGLVQPGGSLRLSCAASGFTFSSYIMMW |
| | | L1) | VRQAPGKGLEWVSSIYPSGGITFYADTVKGRFTISR |
| | | | DNSKNTLYLQMNSLRAEDTAVYYCARIKLGTVTTVD |
| | | | YWGQGTLVTVSS |
| 88 | VH | Durvalumab (PD- | EVQLVESGGGLVQPGGSLRLSCAASGFTFSRYWMSW |
| | | L1) | VRQAPGKGLEWVANIKQDGSEKYYVDSVKGRFTISR |
| | | | DNAKNSLYLQMNSLRAEDTAVYYCAREGGWFGELAF |
| | | | DYWGQGTLVTVSS |
| 89 | VH | Cetuximab | QVQLKQSGPGLVQPSQSLSITCTVSGFSLTNYGVHW |
| | | (EGFR) | VRQSPGKGLEWLGVIWSGGNTDYNTPFTSRLSINKD |
| | | | NSKSQVFFKMNSLQSNDTAIYYCARALTYYDYEFAY |
| | | | WGQGTLVTVSA |
| 90 | VH | Magrolimab | QVQLVQSGAEVKKPGASVKVSCKASGYTFTNYNMHW |
| | | (CD47) | VRQAPGQRLEWMGTIYPGNDDTSYNQKFKDRVTITA |
| | | | DTSASTAYMELSSLRSEDTAVYYCARGGYRAMDYWG |
| | | | QGTLVTVSS |
| 91 | VL | Anti-HLA-G | EIVLTQSPGTLSLSPGERATLSCRASNAVSSSYLAW |
| | | | YQQKPGQAPRLLIYGASYRATGIPDRFSGSGSGTDF |
| | | | TLTISRLEPEDFAVYYCQQHSLYPPTFGGGTKVEIK |
| 92 | VL | Nivolumab (PD-1) | EIVLTQSPATLSLSPGERATLSCRASQSVSSYLAWY |
| | | | QQKPGQAPRLLIYDASNRATGIPARFSGSGSGTDFT |
| | | | LTISSLEPEDFAVYYCQQSSNWPRTFGQGTKVEIK |
| 93 | VL | Pembrolizumab | EIVLTQSPATLSLSPGERATLSCRASKGVSTSGYSY |
| | | (PD-1) | LHWYQQKPGQAPRLLIYLASYLESGVPARFSGSGSG |
| | | | TDFTLTISSLEPEDFAVYYCQHSRDLPLTFGGGTKV |
| | | | EIK |
| 94 | VL | Cemiplimab (PD- | DIQMTQSPSSLSASVGDSITITCRASLSINTFLNWY |
| | | 1) | QQKPGKAPNLLIYAASSLHGGVPSRFSGSGSGTDFT |
| | | | LTIRTLQPEDFATYYCQQSSNTPFTFGPGTVVDFR |
| 95 | VL | Dostarlimab (PD- | DIQLTQSPSFLSAYVGDRVTITCKASQDVGTAVAWY |
| | | 1) | QQKPGKAPKLLIYWASTLHTGVPSRFSGSGSGTEFT |
| | | | LTISSLQPEDFATYYCQHYSSYPWTFGQGTKLEIK |
| 96 | VL | Atezolizumab | DIQMTQSPSSLSASVGDRVTITCRASQDVSTAVAWY |
| | | (PD-L1) | QQKPGKAPKLLIYSASFLYSGVPSRFSGSGSGTDFT |
| | | | LTISSLQPEDFATYYCQQYLYHPATFGQGTKVEIK |
| 97 | VL | Avelumab (PD- | QSALTQPASVSGSPGQSITISCTGTSSDVGGYNYVS |
| | | L1) | WYQQHPGKAPKLMIYDVSNRPSGVSNRFSGSKSGNT |
| | | | ASLTISGLQAEDEADYYCSSYTSSSTRVFGTGTKVT |
| | | | VL |
| 98 | VL | Durvalumab (PD- | EIVLTQSPGTLSLSPGERATLSCRASQRVSSSYLAW |
| | | L1) | YQQKPGQAPRLLIYDASSRATGIPDRFSGSGSGTDF |
| | | | TLTISRLEPEDFAVYYCQQYGSLPWTFGQGTKVEIK |
| 99 | VL | Cetuximab | DILLTQSPVILSVSPGERVSFSCRASQSIGTNIHWY |
| | | (EGFR) | QQRTNGSPRLLIKYASESISGIPSRFSGSGSGTDFT |
| | | | LSINSVESEDIADYYCQQNNNWPTTFGAGTKLELK |
| 100 | VL | Magrolimab | DIVMTQSPLSLPVTPGEPASISCRSSQSIVYSNGNT |
| | | (CD47) | YLGWYLQKPGQSPQLLIYKVSNRFSGVPDRFSGSGS |
| | | | GTDFTLKISRVEAEDVGVYYCFQGSHVPYTFGQGTK |
| | | | LEIK |
| 101 | HC | Anti-HLA-G | QVQLQESGPGLVKPSETLSLTCAVSGYSILSGYYWF |
| | | | WIRQPPGKGLEWIGGIYHSGSTAYNPSLKSRVTISV |
| | | | DTSKNQFSLKLSSVTAADTAVYYCARGGEVTYSRGP |
| | | | LDVWGQGTTVTVSSASTKGPSVFPLAPSSKSTSGGT |
| | | | AALGCLVKDYFPEPVTVSWNSGALTSGVHTFPAVLQ |
| | | | SSGLYSLSSVVTVPSSSLGTQTYICNVNHKPSNTKV |
| | | | DKKVEPKSCDKTHTCPPCPAPEAAGAPSVFLFPPKP |
| | | | KDTLMISRTPEVTCVVVDVSHEDPEVKFNWYVDGVE |
| | | | VHNAKTKPREEQYNSTYRVVSVLTVLHQDWLNGKEY |
| | | | KCKVSNKALPAPIEKTISKAKGQPREPQVYTLPPSR |
| | | | EEMTKNQVSLTCLVKGFYPSDIAVEWESNGQPENNY |
| | | | KTTPPVLDSDGSFFLYSKLTVDKSRWQQGNVFSCSV |
| | | | MHEALHNHYTQKSLSLSPGK |
| 102 | HC | Nivolumab (PD-1) | QVQLVESGGGVVQPGRSLRLDCKASGITFSNSGMHW |
| | | | VRQAPGKGLEWVAVIWYDGSKRYYADSVKGRFTISR |
| | | | DNSKNTLFLQMNSLRAEDTAVYYCATNDDYWGQGTL |
| | | | VTVSSASTKGPSVFPLAPCSRSTSESTAALGCLVKD |
| | | | YFPEPVTVSWNSGALTSGVHTFPAVLQSSGLYSLSS |
| | | | VVTVPSSSLGTKTYTCNVDHKPSNTKVDKRVESKYG |
| | | | PPCPPCPAPEFLGGPSVFLFPPKPKDTLMISRTPEV |
| | | | TCVVVDVSQEDPEVQFNWYVDGVEVHNAKTKPREEQ |
| | | | FNSTYRVVSVLTVLHQDWLNGKEYKCKVSNKGLPSS |
| | | | IEKTISKAKGQPREPQVYTLPPSQEEMTKNQVSLTC |
| | | | LVKGFYPSDIAVEWESNGQPENNYKTTPPVLDSDGS |
| | | | FFLYSRLTVDKSRWQEGNVFSCSVMHEALHNHYTQK |
| | | | SLSLSLGK |
| 103 | HC | Pembrolizumab | QVQLVQSGVEVKKPGASVKVSCKASGYTFTNYYMYW |
| | | (PD-1) | VRQAPGQGLEWMGGINPSNGGTNFNEKFKNRVTLTT |
| | | | DSSTTTAYMELKSLQFDDTAVYYCARRDYRFDMGFD |
| | | | YWGQGTTVTVSSASTKGPSVFPLAPCSRSTSESTAA |
| | | | LGCLVKDYFPEPVTVSWNSGALTSGVHTFPAVLQSS |
| | | | GLYSLSSVVTVPSSSLGTKTYTCNVDHKPSNTKVDK |
| | | | RVESKYGPPCPPCPAPEFLGGPSVFLFPPKPKDTLM |
| | | | ISRTPEVTCVVVDVSQEDPEVQFNWYVDGVEVHNAK |
| | | | TKPREEQFNSTYRVVSVLTVLHQDWLNGKEYKCKVS |
| | | | NKGLPSSIEKTISKAKGQPREPQVYTLPPSQEEMTK |
| | | | NQVSLTCLVKGFYPSDIAVEWESNGQPENNYKTTPP |
| | | | VLDSDGSFFLYSRLTVDKSRWQEGNVFSCSVMHEAL |
| | | | HNHYTQKSLSLSLGK |
| 104 | HC | Cemiplimab (PD- | EVQLLESGGVLVQPGGSLRLSCAASGFTFSNFGMTW |
| | | 1) | VRQAPGKGLEWVSGISGGGRDTYFADSVKGRFTISR |
| | | | DNSKNTLYLQMNSLKGEDTAVYYCVKWGNIYFDYWG |
| | | | QGTLVTVSSASTKGPSVFPLAPCSRSTSESTAALGC |
| | | | LVKDYFPEPVTVSWNSGALTSGVHTFPAVLQSSGLY |
| | | | SLSSVVTVPSSSLGTKTYTCNVDHKPSNTKVDKRVE |
| | | | SKYGPPCPPCPAPEFLGGPSVFLFPPKPKDTLMISR |
| | | | TPEVTCVVVDVSQEDPEVQFNWYVDGVEVHNAKTKP |
| | | | REEQFNSTYRVVSVLTVLHQDWLNGKEYKCKVSNKG |
| | | | LPSSIEKTISKAKGQPREPQVYTLPPSQEEMTKNQV |
| | | | SLTCLVKGFYPSDIAVEWESNGQPENNYKTTPPVLD |
| | | | SDGSFFLYSRLTVDKSRWQEGNVFSCSVMHEALHNH |
| | | | YTQKSLSLSLGK |
| 105 | HC | Dostarlimab (PD- | EVQLLESGGGLVQPGGSLRLSCAASGFTFSSYDMSW |
| | | 1) | VRQAPGKGLEWVSTISGGGSYTYYQDSVKGRFTISR |
| | | | DNSKNTLYLQMNSLRAEDTAVYYCASPYYAMDYWGQ |
| | | | GTTVTVSSASTKGPSVFPLAPCSRSTSESTAALGCL |
| | | | VKDYFPEPVTVSWNSGALTSGVHTFPAVLQSSGLYS |
| | | | LSSVVTVPSSSLGTKTYTCNVDHKPSNTKVDKRVES |
| | | | KYGPPCPPCPAPEFLGGPSVFLFPPKPKDTLMISRT |
| | | | PEVTCVVVDVSQEDPEVQFNWYVDGVEVHNAKTKPR |
| | | | EEQFNSTYRVVSVLTVLHQDWLNGKEYKCKVSNKGL |
| | | | PSSIEKTISKAKGQPREPQVYTLPPSQEEMTKNQVS |
| | | | LTCLVKGFYPSDIAVEWESNGQPENNYKTTPPVLDS |
| | | | DGSFFLYSRLTVDKSRWQEGNVFSCSVMHEALHNHY |
| | | | TQKSLSLSLGK |
| 106 | HC | Atezolizumab | EVQLVESGGGLVQPGGSLRLSCAASGFTFSDSWIHW |
| | | (PD-L1) | VRQAPGKGLEWVAWISPYGGSTYYADSVKGRFTISA |
| | | | DTSKNTAYLQMNSLRAEDTAVYYCARRHWPGGFDYW |
| | | | GQGTLVTVSSASTKGPSVFPLAPSSKSTSGGTAALG |
| | | | CLVKDYFPEPVTVSWNSGALTSGVHTFPAVLQSSGL |
| | | | YSLSSVVTVPSSSLGTQTYICNVNHKPSNTKVDKKV |
| | | | EPKSCDKTHTCPPCPAPELLGGPSVFLFPPKPKDTL |
| | | | MISRTPEVTCVVVDVSHEDPEVKFNWYVDGVEVHNA |
| | | | KTKPREEQYASTYRVVSVLTVLHQDWLNGKEYKCKV |
| | | | SNKALPAPIEKTISKAKGQPREPQVYTLPPSREEMT |
| | | | KNQVSLTCLVKGFYPSDIAVEWESNGQPENNYKTTP |
| | | | PVLDSDGSFFLYSKLTVDKSRWQQGNVFSCSVMHEA |
| | | | LHNHYTQKSLSLSPGK |
| 107 | HC | Avelumab (PD- | EVQLLESGGGLVQPGGSLRLSCAASGFTFSSYIMMW |
| | | L1) | VRQAPGKGLEWVSSIYPSGGITFYADTVKGRFTISR |
| | | | DNSKNTLYLQMNSLRAEDTAVYYCARIKLGTVTTVD |
| | | | YWGQGTLVTVSSASTKGPSVFPLAPSSKSTSGGTAA |
| | | | LGCLVKDYFPEPVTVSWNSGALTSGVHTFPAVLQSS |
| | | | GLYSLSSVVTVPSSSLGTQTYICNVNHKPSNTKVDK |
| | | | KVEPKSCDKTHTCPPCPAPELLGGPSVFLFPPKPKD |
| | | | TLMISRTPEVTCVVVDVSHEDPEVKFNWYVDGVEVH |
| | | | NAKTKPREEQYNSTYRVVSVLTVLHQDWLNGKEYKC |
| | | | KVSNKALPAPIEKTISKAKGQPREPQVYTLPPSRDE |
| | | | LTKNQVSLTCLVKGFYPSDIAVEWESNGQPENNYKT |
| | | | TPPVLDSDGSFFLYSKLTVDKSRWQQGNVFSCSVMH |
| | | | EALHNHYTQKSLSLSPGK |
| 108 | HC | Durvalumab (PD- | EVQLVESGGGLVQPGGSLRLSCAASGFTFSRYWMSW |
| | | L1) | VRQAPGKGLEWVANIKQDGSEKYYVDSVKGRFTISR |
| | | | DNAKNSLYLQMNSLRAEDTAVYYCAREGGWFGELAF |
| | | | DYWGQGTLVTVSSASTKGPSVFPLAPSSKSTSGGTA |
| | | | ALGCLVKDYFPEPVTVSWNSGALTSGVHTFPAVLQS |
| | | | SGLYSLSSVVTVPSSSLGTQTYICNVNHKPSNTKVD |
| | | | KRVEPKSCDKTHTCPPCPAPEFEGGPSVFLFPPKPK |
| | | | DTLMISRTPEVTCVVVDVSHEDPEVKENWYVDGVEV |
| | | | HNAKTKPREEQYNSTYRVVSVLTVLHQDWLNGKEYK |
| | | | CKVSNKALPASIEKTISKAKGQPREPQVYTLPPSRE |
| | | | EMTKNQVSLTCLVKGFYPSDIAVEWESNGQPENNYK |
| | | | TTPPVLDSDGSFFLYSKLTVDKSRWQQGNVFSCSVM |
| | | | HEALHNHYTQKSLSLSPGK |
| 109 | HC | Cetuximab | QVQLKQSGPGLVQPSQSLSITCTVSGFSLTNYGVHW |
| | | (EGFR) | VRQSPGKGLEWLGVIWSGGNTDYNTPFTSRLSINKD |
| | | | NSKSQVFFKMNSLQSNDTAIYYCARALTYYDYEFAY |
| | | | WGQGTLVTVSAASTKGPSVFPLAPSSKSTSGGTAAL |
| | | | GCLVKDYFPEPVTVSWNSGALTSGVHTFPAVLQSSG |
| | | | LYSLSSVVTVPSSSLGTQTYICNVNHKPSNTKVDKK |
| | | | VEPKSCDKTHTCPPCPAPELLGGPSVFLFPPKPKDT |
| | | | LMISRTPEVTCVVVDVSHEDPEVKFNWYVDGVEVHN |
| | | | AKTKPREEQYNSTYRVVSVLTVLHQDWLNGKEYKCK |
| | | | VSNKALPAPIEKTISKAKGQPREPQVYTLPPSRDEL |
| | | | TKNQVSLTCLVKGFYPSDIAVEWESNGQPENNYKTT |
| | | | PPVLDSDGSFFLYSKLTVDKSRWQQGNVFSCSVMHE |
| | | | ALHNHYTQKSLSLSPGK |
| 110 | HC | Magrolimab | QVQLVQSGAEVKKPGASVKVSCKASGYTFTNYNMHW |
| | | (CD47) | VRQAPGQRLEWMGTIYPGNDDTSYNQKFKDRVTITA |
| | | | DTSASTAYMELSSLRSEDTAVYYCARGGYRAMDYWG |
| | | | QGTLVTVSSASTKGPSVFPLAPCSRSTSESTAALGC |
| | | | LVKDYFPEPVTVSWNSGALTSGVHTFPAVLQSSGLY |
| | | | SLSSVVTVPSSSLGTKTYTCNVDHKPSNTKVDKRVE |
| | | | SKYGPPCPPCPAPEFLGGPSVFLFPPKPKDTLMISR |
| | | | TPEVTCVVVDVSQEDPEVQFNWYVDGVEVHNAKTKP |
| | | | REEQFNSTYRVVSVLTVLHQDWLNGKEYKCKVSNKG |
| | | | LPSSIEKTISKAKGQPREPQVYTLPPSQEEMTKNQV |
| | | | SLTCLVKGFYPSDIAVEWESNGQPENNYKTTPPVLD |
| | | | SDGSFFLYSRLTVDKSRWQEGNVFSCSVMHEALHNH |
| | | | YTQKSLSLSLGK |
| 111 | LC | Anti-HLA-G | EIVLTQSPGTLSLSPGERATLSCRASNAVSSSYLAW |
| | | | YQQKPGQAPRLLIYGASYRATGIPDRFSGSGSGTDF |
| | | | TLTISRLEPEDFAVYYCQQHSLYPPTFGGGTKVEIK |
| | | | RTVAAPSVFIFPPSDEQLKSGTASVVCLLNNFYPRE |
| | | | AKVQWKVDNALQSGNSQESVTEQDSKDSTYSLSSTL |
| | | | TLSKADYEKHKVYACEVTHQGLSSPVTKSENRGEC |
| 112 | LC | Nivolumab (PD-1) | EIVLTQSPATLSLSPGERATLSCRASQSVSSYLAWY |
| | | | QQKPGQAPRLLIYDASNRATGIPARFSGSGSGTDFT |
| | | | LTISSLEPEDFAVYYCQQSSNWPRTFGQGTKVEIKR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSENRGEC |
| 113 | LC | Pembrolizumab | EIVLTQSPATLSLSPGERATLSCRASKGVSTSGYSY |
| | | (PD-1) | LHWYQQKPGQAPRLLIYLASYLESGVPARFSGSGSG |
| | | | TDFTLTISSLEPEDFAVYYCQHSRDLPLTFGGGTKV |
| | | | EIKRTVAAPSVFIFPPSDEQLKSGTASVVCLLNNFY |
| | | | PREAKVQWKVDNALQSGNSQESVTEQDSKDSTYSLS |
| | | | STLTLSKADYEKHKVYACEVTHQGLSSPVTKSFNRG |
| | | | EC |
| 114 | LC | Cemiplimab (PD- | DIQMTQSPSSLSASVGDSITITCRASLSINTFLNWY |
| | | 1) | QQKPGKAPNLLIYAASSLHGGVPSRFSGSGSGTDFT |
| | | | LTIRTLQPEDFATYYCQQSSNTPFTFGPGTVVDFRR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSFNRGEC |
| 115 | LC | Dostarlimab (PD- | DIQLTQSPSFLSAYVGDRVTITCKASQDVGTAVAWY |
| | | 1) | QQKPGKAPKLLIYWASTLHTGVPSRFSGSGSGTEFT |
| | | | LTISSLQPEDFATYYCQHYSSYPWTFGQGTKLEIKR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSFNRGEC |
| 116 | LO | Atezolizumab | DIQMTQSPSSLSASVGDRVTITCRASQDVSTAVAWY |
| | | (PD-L1) | QQKPGKAPKLLIYSASFLYSGVPSRFSGSGSGTDFT |
| | | | LTISSLQPEDFATYYCQQYLYHPATFGQGTKVEIKR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSENRGEC |
| 117 | LC | Avelumab (PD- | QSALTQPASVSGSPGQSITISCTGTSSDVGGYNYVS |
| | | L1) | WYQQHPGKAPKLMIYDVSNRPSGVSNRFSGSKSGNT |
| | | | ASLTISGLQAEDEADYYCSSYTSSSTRVFGTGTKVT |
| | | | VLGQPKANPTVTLFPPSSEELQANKATLVCLISDFY |
| | | | PGAVTVAWKADGSPVKAGVETTKPSKQSNNKYAASS |
| | | | YLSLTPEQWKSHRSYSCQVTHEGSTVEKTVAPTECS |
| 118 | LC | Durvalumab (PD- | EIVLTQSPGTLSLSPGERATLSCRASQRVSSSYLAW |
| | | L1) | YQQKPGQAPRLLIYDASSRATGIPDRFSGSGSGTDF |
| | | | TLTISRLEPEDFAVYYCQQYGSLPWTFGQGTKVEIK |
| | | | RTVAAPSVFIFPPSDEQLKSGTASVVCLLNNFYPRE |
| | | | AKVQWKVDNALQSGNSQESVTEQDSKDSTYSLSSTL |
| | | | TLSKADYEKHKVYACEVTHQGLSSPVTKSFNRGEC |
| 119 | LC | Cetuximab | DILLTQSPVILSVSPGERVSFSCRASQSIGTNIHWY |
| | | (EGFR) | QQRTNGSPRLLIKYASESISGIPSRFSGSGSGTDFT |
| | | | LSINSVESEDIADYYCQQNNNWPTTFGAGTKLELKR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSENRGEC |
| 120 | LC | Magrolimab | DIVMTQSPLSLPVTPGEPASISCRSSQSIVYSNGNT |
| | | (CD47) | YLGWYLQKPGQSPQLLIYKVSNRFSGVPDRFSGSGS |
| | | | GTDFTLKISRVEAEDVGVYYCFQGSHVPYTFGQGTK |
| | | | LEIKRTVAAPSVFIFPPSDEQLKSGTASVVCLLNNF |
| | | | YPREAKVQWKVDNALQSGNSQESVTEQDSKDSTYSL |
| | | | SSTLTLSKADYEKHKVYACEVTHQGLSSPVTKSFNR |
| | | | GEC |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

1. A method for treatment of a subject suffering from cancer, comprising administering to the subject: an antibody which binds to HLA-G and an antibody which binds to an epidermal growth factor receptor (EGFR).

2. The method of claim 1, wherein the antibody that binds to HLA-G comprises or consists of a heavy chain variable region (VH) and a light chain variable region (VL), with VH and/or VL comprising:
a) a VHCDR1 having the sequence set forth in any one of SEQ ID NO: 1 or SEQ ID NO: 11,
b) a VHCDR2 having the sequence set forth in any one of SEQ ID NO: 21 or SEQ ID NO: 31,
c) a VHCDR3 having the sequence set forth in any one of SEQ ID NO: 41,
d) a VLCDR1 having the sequence set forth in any one of SEQ ID NO: 51,
e) a VLCDR2 having the sequence set forth in any one of SEQ ID NO: 61, and
f) a VLCDR3 having the sequence set forth in any one of SEQ ID NO: 71.

3. The method of claim 1 or claim 2, wherein the antibody that binds to HLA-G comprises or consists of a heavy chain variable region (VH) and a light chain variable region (VL), with VH comprising, consisting of, or consisting essentially of a SEQ ID NO: 81 and with VL comprising, consisting of, or consisting essentially of a VL having the sequence set forth in SEQ ID NO: 91.

4. The method of any of claims 1-3, wherein the antibody which binds to an epidermal growth factor receptor (EGFR) comprises or consists of a heavy chain variable region (VH) and a light chain variable region (VL), VH and/or VL comprising:
a) a VHCDR1 having the sequence set forth in SEQ ID NO: 9 or SEQ ID NO: 19,
b) a VHCDR2 having the sequence set forth in SEQ ID NO: 29 or SEQ ID NO: 39,
c) a VHCDR3 having the sequence set forth in SEQ ID NO: SEQ ID NO: 49,
d) a VLCDR1 having the sequence set forth in SEQ ID NO: SEQ ID NO: 59,
e) a VLCDR2 having the sequence set forth in SEQ ID NO: SEQ NO: 69, and
f) a VLCDR3 having the sequence set forth in SEQ ID NO: 79.

5. The method of any of claims 1-4, wherein the antibody which binds to an epidermal growth factor receptor (EGFR) comprises or consists of a heavy chain variable region (VH) and a light chain variable region (VL), VH and/or VL comprising, consisting of, or consisting essentially of a VH having the sequence set forth in SEQ ID NO: 89 and of a VL comprising, consisting of, or consisting essentially of a VL having the sequence set forth in SEQ ID NO: 99.

6. A method for treatment of a subject suffering from cancer, comprising administering to the subject: an antibody which binds to HLA-G and an antibody which binds to PD-1 or PD-L1.

7. The method of claim 6, wherein the antibody that binds to HLA-G comprises or consists of a heavy chain variable region (VH) and a light chain variable region (VL), with VH and/or VL comprising:
a) a VHCDR1 having the sequence set forth in any one of SEQ ID NO: 1 or SEQ ID NO: 11,
b) a VHCDR2 having the sequence set forth in any one of SEQ ID NO: 21 or SEQ ID NO: 31,
c) a VHCDR3 having the sequence set forth in any one of SEQ ID NO: 41,
d) a VLCDR1 having the sequence set forth in any one of SEQ ID NO: 51,
e) a VLCDR2 having the sequence set forth in any one of SEQ ID NO: 61, and
f) a VLCDR3 having the sequence set forth in any one of SEQ ID NO: 71.

8. The method of claim 6 or claim 7, wherein the antibody that binds to HLA-G comprises or consists of a heavy chain variable region (VH) and a light chain variable region (VL), with VH comprising, consisting of, or consisting essentially of SEQ ID NO: 81 and with VL comprising, consisting of, or consisting essentially of a VL having the sequence set forth in SEQ ID NO: 91.

9. The method of any of claims 6-8, wherein the an antibody which binds to PD-1 and/or PD-L1 comprises or consists of a heavy chain variable region (VH) and a light chain variable region (VL), VH and/or VL comprising:
a) a VHCDR1 having the sequence set forth in SEQ ID NOs: 2-8 or SEQ ID NOs: 12-18,
b) a VHCDR2 having the sequence set forth in SEQ ID NOs: 22-28 or SEQ NOs 32-38,
c) a VHCDR3 having the sequence set forth in SEQ ID NOs: 42-48,
d) a VLCDR1 having the sequence set forth in SEQ ID NOs: 52-58,
e) a VLCDR2 having the sequence set forth in SEQ ID NOs: 62-68, and
f) a VLCDR3 having the sequence set forth in SEQ ID NOs: 72-78.

10. The method of any of claims 6-9, wherein the antibody which binds to PD-1 and/or PD-L1 comprises or consists of a heavy chain variable region (VH) and a light chain variable region (VL), VH and/or VL comprising, consisting of, or consisting essentially of a VH having the sequence set forth in SEQ ID NOs: 82-88 and a VL comprising, consisting of, or consisting essentially of a VL having the sequence set forth in SEQ ID NOs: 92-98.

11. A method for treatment of a subject suffering from cancer, comprising administering to the subject: an antibody which binds to HLA-G and an antibody which binds to CD47.

12. The method of claim 11, wherein the antibody that binds to HLA-G comprises or consists of a heavy chain variable region (VH) and a light chain variable region (VL), with VH and/or VL comprising:
a) a VHCDR1 having the sequence set forth in any one of SEQ ID NO: 1 or SEQ ID NO: 11,
b) a VHCDR2 having the sequence set forth in any one of SEQ ID NO: 21 or SEQ ID NO: 31,
c) a VHCDR3 having the sequence set forth in any one of SEQ ID NO: 41,
d) a VLCDR1 having the sequence set forth in any one of SEQ ID NO: 51,
e) a VLCDR2 having the sequence set forth in any one of SEQ ID NO: 61, and
f) a VLCDR3 having the sequence set forth in any one of SEQ ID NO: 71.

13. The method of claim 11 or claim 12, wherein the antibody that binds to HLA-G comprises or consists of a heavy chain variable region (VH) and a light chain variable region (VL), with VH comprising, consisting of, or consisting essentially of a SEQ ID NO: 81 and with VL comprising, consisting of, or consisting essentially of a VL having the sequence set forth in SEQ ID NO: 91.

14. The method of any of claims 11-13, wherein the antibody which binds to an CD47 comprises or consists of a heavy chain variable region (VH) and a light chain variable region (VL), VH and/or VL comprising:
a) a VHCDR1 having the sequence set forth in SEQ ID NO: 10 or SEQ ID NO: 20,
b) a VHCDR2 having the sequence set forth in SEQ ID NO: 30 or SEQ ID NO: 40,
c) a VHCDR3 having the sequence set forth in SEQ ID NO: 50,
d) a VLCDR1 having the sequence set forth in SEQ ID NO: 60,
e) a VLCDR2 having the sequence set forth in SEQ ID NO: 70, and
f) a VLCDR3 having the sequence set forth in SEQ ID NO: 80.

15. The method of any of claims 11-14, wherein the antibody which binds to CD47 comprises or consists of a heavy chain variable region (VH) and a light chain variable region (VL), VH and/or VL comprising, consisting of, or consisting essentially of a VH having the sequence set forth in SEQ ID NO: 90 and of a VL comprising, consisting of, or consisting essentially of a VL having the sequence set forth in SEQ ID NO: 100.

16. A pharmaceutical composition for use in the method of any of the above claims.

17. The method of any of claims 1-15, wherein the cancer is a solid cancer.

18. The method of any of claims 1-15, wherein the cancer is a hematological cancer.

19. The method of any of claims 1-15, wherein the subject is a human subject.

20. The method of any of claims 1-15, wherein the pharmaceutical composition is administered in an amount sufficient to achieve 1, 2, 3, 4, 5, 6, 7, or 8 of the following in the subject:
a) inhibition of immune suppression;
b) reduction of levels of regulatory T cells;
c) increase in activity of myeloid cells, cytotoxic T lymphocytes, helper T cells, NK cells, T cells, B cells, neutrophils, monocytes, macrophages, and/or dendritic cells;
d) increase in phagocytic activity;
e) inhibition of metastasis;
f) inhibition of tumor growth;
g) induction of tumor regression; and/or
h) enhancement of ILT2⁺ CD8⁺ T cell degranulation.

21. The method of claim 20, wherein the increase in phagocytic activity comprises an increase in percent phagocytosis as compared to a pharmaceutical composition with no antibody or a pharmaceutical composition with an antibody which binds HLA-G.

22. The method of any of claims 1-15, wherein the method further comprises one or more of the following:
a) administering chemotherapy;
b) administering radiation therapy; and/or
c) administering one or more additional therapeutic agents.
